(19) United States
(12) Patent Application Publication
(10) Pub. No.: US 2009/0062317 A1
(43) Pub. Date: Mar. 5, 2009

(54) MEDICINAL USE OF RECEPTOR LIGANDS

(75) Inventors: Jean-Marie Receveur, Hoersholm (DK); Emelie Bjurling, Hoersholm (DK); Ann Christensen, Hoersholm (DK); Thomas Hoegberg, Hoersholm (DK)

Correspondence Address: BANNER & WITCOFF, LTD., 1100 13th STREET, N.W., SUITE 1200, WASHINGTON, DC, 20005-4051, US

(73) Assignee: 7TM PHARMA A/S, Hoersholm (DK)

(21) Appl. No.: 11/658,307

(22) PCT Filed: Jul. 5, 2005
(86) PCT No.: PCT/EP2005/007318

§ 371 Date: Oct. 31, 2008

(30) Foreign Application Priority Data
Jul. 27, 2004 (GB) 0416728.4

(51) Int. Cl. A61K 31/517; C07D 401/12; A61K 31/454; A61K 31/4709

(52) U.S. Cl. 514/266.22; 544/292; 514/322; 546/273.4; 546/163; 514/313

(57) ABSTRACT

Compounds of formula (I) are ligands of the melanin concentrating hormone-1 receptor (MCH-1R), useful in the treatment of diseases responsive to modulation of melanin concentrating hormone (MCH) activity, for example feeding disorders and diseases for which obesity is a risk factor (I): wherein ring B is selected from specific substituted phenyl or benz-fused 5 membered N-containing heterocycles defined in the specification; R, is attached to a ring carbon of ring B, and represents hydrogen, F, Cl, or —OCH₃; X is ═CH— or ═N—; L, is —CH₂— or —CH₂CH₂—; L₂ is a bond, —CH₂— or —CO—; R2 is H or C, —C₃ alkyl, or —N(R₂) L, —is selected from specific cyclic amino linker radicals as defined in the specification; ring A is selected from specific N-containing heterocyclic rings as defined in the specification.

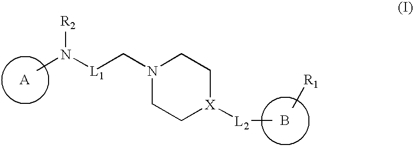

This invention relates to the use of a class of compounds which are ligands of the melanin concentrating hormone-1 receptor (MCH-1R), in the treatment of diseases responsive to modulation of melanin concentrating hormone (MCH) activity, for example feeding disorders; diseases for which obesity is a risk factor such as metabolic syndrome, Type II diabetes, cardiovascular disease, osteoarthritis, and some cancers; mental disorders; sexual dysfunctions; reproductive dysfunctions; and epilepsy. The invention also relates to novel members of that class of compounds, to pharmaceutical compositions containing them, and to the use of the compounds in combination with other treatments for MHC-dependent diseases.

### BACKGROUND TO THE INVENTION

Obesity has become a global epidemic with a steadily increasing prevalence not only confined to the industrialized countries (Kopelman. Obesity as a medical problem. Nature 2000, 404, 635-643; International Association for the Study of Obesity (IASO) www.iaso.org). Thus, obesity is no longer regarded as a cosmetic problem but a major contributor to the development of diseases including Type II diabetes, coronary heart disease, certain forms of cancer, osteoarthritis and sleep apnoea.

Several approaches for treatment of obesity can be envisioned—i) Reducing food intake ii) Preventing fat absorption iii) Increasing thermogenesis iv) Modulating fat metabolism or storage v) Modulating central control of body weight—and it is reasonable to expect future therapy to rely upon a combination of modalities (Bray and Tartaglia. Medicinal strategies in the treatment of obesity. Nature 2000, 404, 672-677).

The increasing understanding of central control mechanisms, especially hypothalamic neuropeptide pathways, has provided novel potential targets for drug discovery (Fernández-López et al. Pharmacological approaches for the treatment of obesity. Drugs 2002, 62, 915-944). Thus, the orexigenic (increasing food intake) peptides agouti-related protein (Agrp), neuropeptide Y (NPY), melanin-concentrating hormone (MCH), ghrelin and endocannabinoids have been implicated in food intake and energy homeostasis (Schwartz, et al. Central nervous system control of food intake. Nature 2000, 404, 661-471; Dhillo and Bloom. Hypothalamic peptides as targets for obesity, Curr. Opinion Pharmacol. 2001, 1, 651-655).

MCH is a nonadecapeptide found in rat and human brain, expressed in particular in the lateral hypothalamus. MCH has been implicated in several behavioural and hormonal functions in the rat, including food intake, sexual behaviour, responses to novelty, anxiolysis, the thyroid axis, the gonadal axis and the adrenal axis (Forray. The MCH receptor family: feeding brain disorders? Curr. Opin. Pharmacol. 2003 3, 85-89; Hervieu. Melanin-concentrating hormone functions in the nervous system: food intake and stress. Expert Opin. Ther. Targets 2003, 7, 495-511; Kawano et al. Melanin-concentrating hormone neuron system: the wide web that controls the feeding. Anat. Sci. Int. 2002, 77, 149-160; Butler and Cone. Knockout models resulting in the development of obesity. Trends Genetics 2001, 17, S50-S53; Kennedy et al. Effect of Direct Injection of Melanin-Concentrating Hormone into the Paraventricular Nucleus Further Evidence for a Stimulatory Role in the Adrenal Axis via SLC-1. J. Neuroendocrinology, 2003, 15, 268-272).

Two seven transmembrane (7TM) G-protein coupled receptors, MCH-1R (SLC-1) and MCH-2R (SLT), have been identified for MCH (Identification of MCH1: Chambers et al. Melanin-concentrating hormone is the cognate ligand for the orphan G protein-coupled receptor SLC-1. Nature 1999, 400, 261-265; Saito et al Molecular characterization of the melanin-concentrating-hormone receptor. Nature 1999, 400, 265-269; Identification of MCH2: Sailer et al. Identification and characterization of a second melanin-concentrating hormone receptor, MCH-2R. Proc. Natl. Acad. Sci. U.S.A. 2001, 98, 7564-7569; An et al. Identification and characterization of a melanin concentrating hormone receptor. Proc. Natl. Acad. Sci. U.S.A. 2001, 98, 7576-7581).

For example, up-regulated MCH mRNA is observed in fasting rats and in obese ob/ob rats, and food consumption is increased upon i.c.v.-injection of MCH in rats (Presse et al. Melanin-concentrating hormone is a potent anorectic peptide regulated by food-deprivation and glucopenia in the rat. Neuroscience 1996, 71, 735-745; Qu et al. A role for melanin-concentrating hormone in the central regulation of feeding behaviour. Nature 1996, 380, 243-247; Rossi et al. Melanin-concentrating hormone acutely stimulates feeding, but chronic administration has no effect on body weight. Endocrinology 1997, 138, 351-355). Also, deletion of the MCH-gene in mice results in a lean phenotype (Shimada et al. Mice lacking melanin-concentrating hormone are hypophagic and lean. Nature 1998, 396, 670-674), whereas overexpression of the MCH-gene in the lateral hypothalamus gives an obese and insulin resistant phenotype (Ludwig et al. Melanin-concentrating hormone overexpression in transgenic mice leads to obesity and insulin resistance. J. Clin. Investig. 2001, 107, 379-386).

MCH-1R knockout mice are reported to be lean, hyperactive and hyperphagic (Marsh et al. Melanin-concentrating hormone 1 receptor-deficient mice are lean, hyperactive, and hyperphagic and have altered metabolism Proc. Natl. Acad. Sci. U.S.A. 2002, 99, 3240-3245).

The distribution of MCH neural systems in rat and human brain implicate a role of modulators of the MCH-1R also in various mental and psychiatric disorders. Thus, MCH-containing fibres project to the isocortex, olfactory regions, hippocampus, amygdala, septum, basal ganglia, thalamus, brainstem, cerebellum and spinal cord (Bittencourt et al. The melanin concentrating hormone system of the rat brain: an immuno- and hybridization histochemical characterization. J. Comparative Neurology 1992, 319, 218-245). The MCH-1 receptor also has a widespread pattern of expression correlating with the distribution of MCH axons throughout the CNS (Kilduff & De Lecea. Mapping of the mRNAs for the hypocretin/orexin and melanin-concentrating hormone receptors: networks of overlapping peptide systems. J. Comparative Neurology 2001 435, 1-5; Saito et al. Expression of the melanin-concentrating hormone (MCH) receptor mRNA in the rat brain. J. Comparative Neurology 2001, 435, 26-40)

For example, the hormone is reported to be involved in memory functions (Monzon et al. Melanin-concentrating hormone (MCH) modifies memory retention in rats. Peptides 1999, 20, 1517-1519; Varas et al. Melanin-concentrating hormone, hippocampal nitric oxide levels and memory retention. Peptides 2002, 23, 2213-2221), anxiety (Monzon and De Barioglio. Response to novelty after i.c.v. injection of melanin-concentrating hormone (MCH) in Rats. Physiol. Behav. 1999, 67, 813-817; Kela et al. Behavioural analysis of melanin-concentrating hormone in rats: evidence for orexigenic and anxiolytic properties Regulatory Peptides 2002, 114, 109-114) and depression (Borowsky et al. Antidepressant, anxiolytic and anorectic effects of a melanin-concentrating hormone-1 receptor antagonist. Nature Med. 2002, 8, 825-830).

MCH expression levels show sensitivity to oestrogenic steroids (Viale et al. 17 beta-estradiol regulation of melanin-concentrating hormone and neuropeptide-E-I contents in cynomolgus monkeys: a preliminary study.

Peptides 1999, 20, 553-559) and MCH stimulates the release of gonadotropin-releasing hormone and gonadotropins in the female rat (Chiocchio et al. Melanin-concentrating hormone stimulates the release of luteinizing hormone-releasing hormone and gonadotropins in the female rat acting at both median eminence and pituitary levels. Biol. Reprod. 2001, 64, 1466-1472), indicating an involvement in the reproductive axis.

Aminotetraline T-226296 was the first reported small molecule MCH-1 receptor antagonist (Takekawa et al. T-226296: a novel, orally active and selective melanin-concentrating hormone receptor antagonist. Eur. J. Pharmacol. 2002, 438, 129-135). T-226296 and SNAP-7941 ((Borowsky et al. Antidepressant, anxiolytic and anorectic effects of a melanin-concentrating hormone-1 receptor antagonist. Nature Med. 2002, 8, 825830) were reported to suppress food intake induced by i.c.v.-injected MCH in rats. Furthermore, a selective peptide antagonist has been reported to reduce food intake and body weight gain after chronic administration (Bednarek et al. Synthesis and Biological Evaluation in Vitro of Selective, High Affinity Peptide Antagonists of Human Melanin-Concentrating Hormone Action at Human Melanin-Concentrating Hormone Receptor 1. Biochemistry 2002, 41, 6383-6390; Shearman et al. Chronic MCH-1 receptor modulation alters appetite, body weight and adiposity in rats. Eur. J. Pharmacol. 2003, 475, 37-47. Note: the peptide sequence for the antagonist (compound B) is erroneous in the latter reference).

Recently, a genetic investigation of subjects with severe early onset obesity revealed two missense variants in MCH-1R which were not found in normal weight controls. (Gibson et al. Melanin-Concentrating Hormone Receptor Mutations and Human Obesity: Functional Analysis. Obesity Res. 2004, 12, 743-749). One of them (R248Q) cosegregated with obesity across two generations.

Thus, MCH-1R modulators, and in particular antagonists, are interesting agents for treatment of metabolic or obesity-related disorders, as well as of various mental or psychiatric disorders.

International patent applications WO 03/004027 and WO 2004/004714, and U.S. Pat. No. 6,727,264 relate to compounds which are antagonists of MCH-1R, in the context of their use for, inter alia, appetite suppression, relief of depression or anxiety, and urinary tract disorders.

International patent application WO 03/045920 also relates to compounds which are antagonists of MCH-1R, in the context of their use for treatment or prevention of obesity, diabetes, appetite and eating disorders, cardiovascular disease, hypertension, dyslipidemia, myocardial infarction, gall stones, osteoarthritis, certain cancers, AIDS wasting, cachexia, frailty, binge eating disorders including bulimia, anorexia, mental disorders including manic depression, depression, schizophrenia, mood disorders, delirium, dementia, severe mental retardation, anxiety, stress, cognitive disorders, sexual function, reproductive function, kidney function, diuresis, locomotor disorders, attention deficit disorder, substance abuse disorders, and dyskinesias including Parkinson's disease, Parkinson-like syndromes, Tourette's syndrome, Huntington's disease, epilepsy, improving memory function, and spinal muscular atrophy.

### DESCRIPTION OF THE INVENTION

The present invention makes available alternative ligands of MHC-1R to those referred to in the literature. The compounds of the invention are believed to be novel per se. The following references disclose specific compounds which are structurally distinct from those of the present invention: U.S. Pat. Nos. 6,727,264, 6,284,759, 5,972,945, International patent applications WO 2004/026828, WO 99/40068, WO 97/31637, WO 95/25726, WO 03004027, Japanese patent application 62051672 and French patent application 2162106.

According to a broad aspect of the present invention there is provided a compound of formula (I), or a salt, hydrate or solvate thereof:

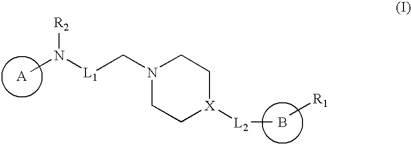

wherein

ring B is selected from

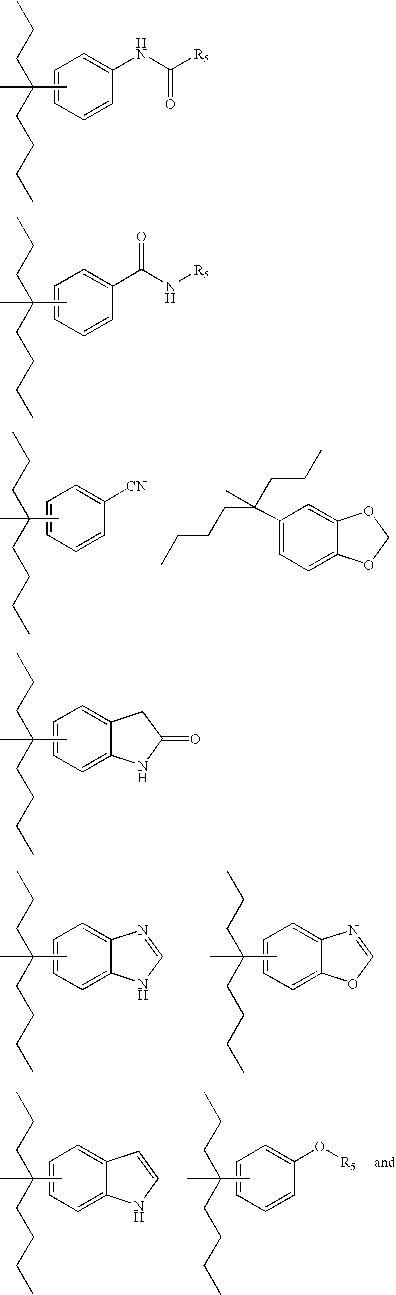

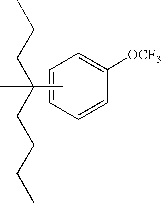

wherein R₅ is C₁-C₄ alkyl or cyclopropyl;

R₁ is attached to a ring carbon of ring B, and represents hydrogen, F, Cl, or —OCH₃;

X is ═CH— or ═N—;

L₁ is —CH₂— or —CH₂CH₂—;

L₂ is a bond, —CH₂— or —CO—;

R₂ is H or C₁-C₃ alkyl, or —N(R₂)L₁- is selected from

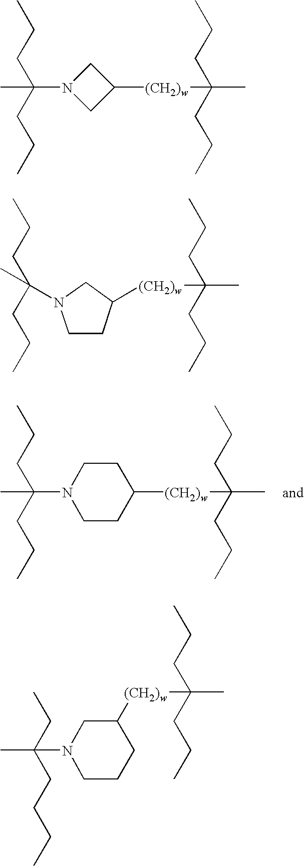

wherein w is 0 or 1;

ring A is selected from

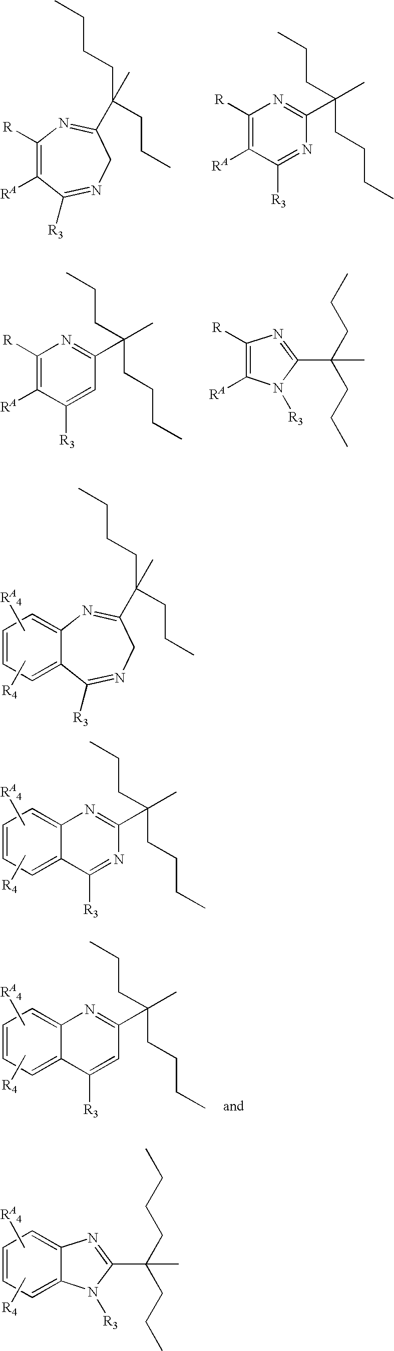

wherein R and R^{A} independently represent hydrogen, C₁-C₄ alkyl, C₁-C₄ alkoxy, halogen, —SCF₃, —OCF₃, or —CF₃;
R₄ and R^{A}₄ independently represent hydrogen, methyl, ethyl, methoxy, F, Cl, —CN, —OCF₃, —CF₃, —CONHCH₃, or —NHCOCH₃; or R₄ and R^{A}₄ together represent —O—CH₂—O— wherein the oxygens are attached to adjacent ring carbons; and
R₃ is a radical of formula -(Z)ₘ-(Alk¹)ₚ-Q wherein
   Q is an optionally substituted monocyclic carbocyclic or heterocyclic ring of 5-, 6- or 7-ring atoms;
   m and p are independently 0 or 1,
   Alk¹ is optionally substituted straight or branched chain divalent C₁-C₃ alkylene radical which may contain a compatible —O—, —S— or —NR₇— link wherein R₇ is hydrogen, methyl, ethyl or n- or iso-propyl, and
   Z is —O— or —NR₈—, wherein R₈ is hydrogen, methyl, ethyl or n- or iso-propyl.

PROVIDED THAT ring A is not

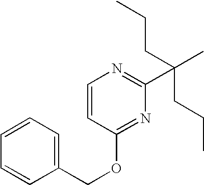

when R₂ is H, L1 is —CH₂CH₂—, X is ═N—, L₂ is a bond, ring B is phenyl, and R₁ is o-methoxy.

The proviso above excludes a compound disclosed as an intermediate on a synthesis disclosed in JP62-61672, CAS 108381-29-9.

The invention also includes pharmaceutical compositions comprising a compound formula (I) or a salt, hydrate or solvate thereof together with a pharmaceutically acceptable carrier.

### TERMINOLOGY

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “divalent (Cₐ-C_{b})alkylene radical” wherein a and b are integers refers to a saturated hydrocarbon chain having from a to b carbon atoms and two unsatisfied valences.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tricyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₁-C₆)alkoxy, hydroxy, hydroxy(C₁-C₆)alkyl, mercapto, mercapto(C₁-C₆)alkyl, (C₁-C₆)alkylthio, halo (including fluoro, bromo and chloro), fully or partially fluorinated (C₁-C₃)alkyl, (C₁-C₃)alkoxy or (C₁-C₃)alkylthio such as trifluoromethyl, trifluoromethoxy, and trifluoromethylthio, nitro, nitrile (—CN), oxo, phenyl, phenoxy, monocyclic heteroaryl or heteroaryloxy with 5 or 6 ring atoms, —COOR^{A}, —COR^{A}, —OCOR^{A}, —SO₂R^{A}, —CONR^{A}R^{B}, —SO₂NR^{A}R^{B}, —NR^{A}R^{B}, OCONR^{A}R^{B}, —NR^{B}COR^{A}, —NR^{B}COOR^{A}, —NR^{B}SO₂OR^{A} or —NR^{A}CONR^{A}R^{B} wherein R^{A} and R^{B} are independently hydrogen or a (C₁-C₆)alkyl group or, in the case where R^{A} and R^{B} are linked to the same N atom, R^{A} and R^{B} taken together with that nitrogen may form a cyclic amino ring. Where the substituent is phenyl, phenoxy or monocyclic heteroaryl or heteroaryloxy with 5 or 6 ring atoms, the phenyl or heteroaryl ring thereof may itself be substituted by any of the above substituents except phenyl phenoxy, heteroaryl or heteroaryloxy. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

For a review on suitable salts, see Handbook of Pharmaceutical Salts: Properties, Selection, and Use by Stahl and Wermuth (Wiley-VCH, Weinheim, Germany, 2002).

The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof.

The compounds of the invention include compounds of formula (I) as hereinbefore defined, including all polymorphs and crystal habits thereof, prodrugs and isomers thereof (including optical, geometric and tautomeric isomers) as hereinafter defined and isotopically-labeled compounds of formula (I).

So-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Also included within the scope of the invention are metabolites of compounds of formula (I), that is, compounds formed in vivo upon administration of the drug. Some examples of metabolites include
(i) where the compound of formula I contains a methyl group, an hydroxymethyl derivative thereof (—CH₃->—CH₂OH):
(ii) where the compound of formula I contains an alkoxy group, an hydroxy derivative thereof (—OR->—OH);
(iii) where the compound of formula I contains a tertiary amino group, a secondary amino derivative thereof (—NR¹R²->—NHR¹ or —NHR²);
(iv) where the compound of formula I contains a secondary amino group, a primary derivative thereof (—NHR¹->—NH₂);
(v) where the compound of formula I contains a phenyl moiety, a phenol derivative thereof (-Ph->-PhOH); and
(vi) where the compound of formula I contains an amide group, a carboxylic acid derivative thereof (—CONH₂->COOH).

### Structural Aspects

For use in accordance with the invention, the following structural characteristics are currently contemplated, in any compatible combination, in the compounds (I):
Of the permitted alternatives for ring A, those of formula (IA), (IB) or (IC), or (ID), (IE) or (IF) are presently preferred:

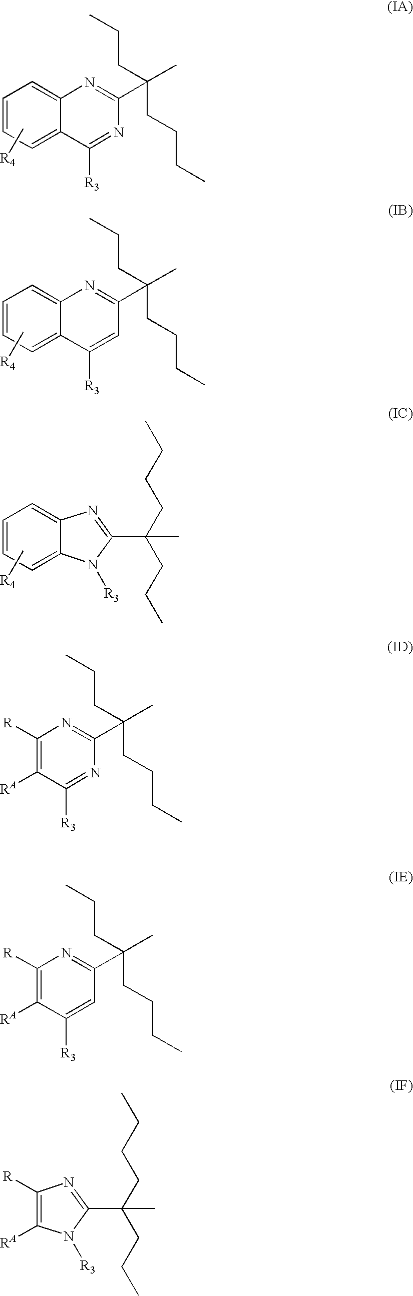

In ring A, R^{A} and R^{A}₄ may be hydrogen; but often neither R nor R^{A} is hydrogen.
Of the permitted alternatives for ring B those of formula (IG), (IH) or (IK) are presently preferred:

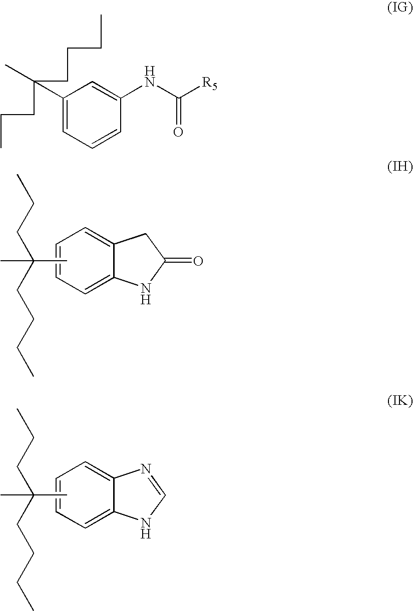

In ring B, R₅ may be, for example methyl, ethyl, n- or iso-propyl, n-, sec- or tert-butyl, but methyl is currently preferred;
Of the alternatives for the linker radical L₂, a bond is currently preferred.
R₂ may be for example hydrogen, methyl, ethyl or n- or iso-propyl, but of the permitted alternatives hydrogen is often preferred; in such cases, the linker radical L₁ may be —CH₂— or CH₂CH₂—, with the latter being currently preferred. R₂ may also form a ring with L1 as specified in the main definition of compounds (I), for example the radical —N(R₂)L₁- may be one of formula:

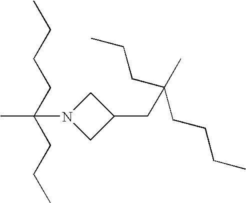

In the R₃ radical -(Z)ₘ-(Alk¹)ₚ-Q in ring A:
   m and p may both be 0; or m may be 1 while p is 0; or m may be 0 while p is 1 and -Alk¹- is —CH₂—; or m may be 0 while p is 1 and Alk¹ is —C(═O)—.
   when m is 1 and p is 0, suitable rings A include

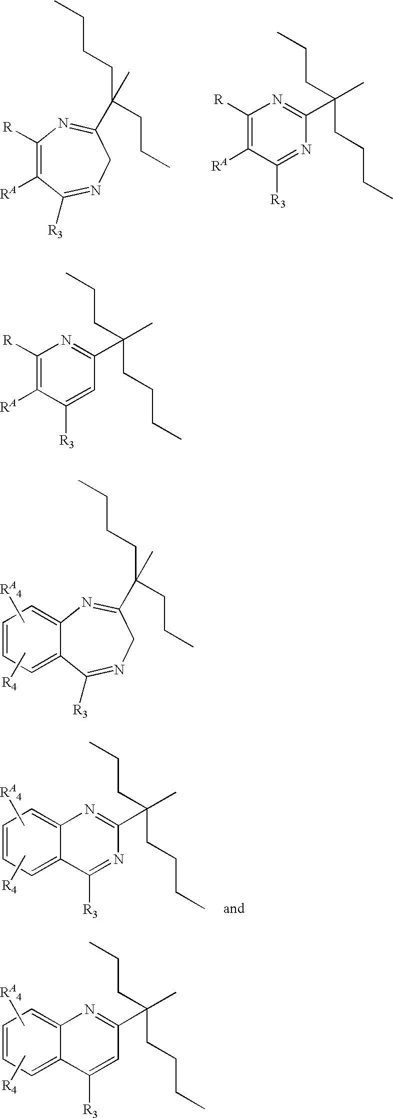

Ring Q may be aromatic or non aromatic. Examples of no aromatic rings Q include N-piperidinyl, N-piperazinyl, and N-morpholinyl. However, it is currently preferred that Q is an optionally substituted aryl or heteroaryl ring, for example an optionally substituted phenyl, pyridyl, or thienyl ring, with optionally substituted phenyl ring often being preferred. Optional substituents may be selected from, for example, fluoro, chloro, methyl, —CN, —OCF₃, —CF₃, —SCH₃, —SO₂CH₃, —SO₂NH₂, —SO₂NHCH₃, —CONHCH₃ or methoxy. In many cases Q will be phenyl, mono-substituted in the 4-position by fluoro, chloro, methyl, —CN, —OCF₃, —CF₃, —SCH₃, —SO₂CH₃, —SO₂NH₂, —SO₂NHCH₃, —CONHCH₃ or methoxy.
In one preferred subclass of compounds (I) of the invention, in the group R₃, Q is optionally substituted phenyl as discussed and specified in the preceding paragraph, and m and p are both 0, or m is 0 and p is 1 and Alk¹ is —CH₂—.

Compounds having one of the following three structural formulae wherein r is 0 or 1, and R₆ is fluoro, chloro, methyl, —CN, —OCF₃, —CF₃ or methoxy, particularly where R₆ is in the 4-position of the phenyl ring, are interesting sub-classes of compounds of the invention:

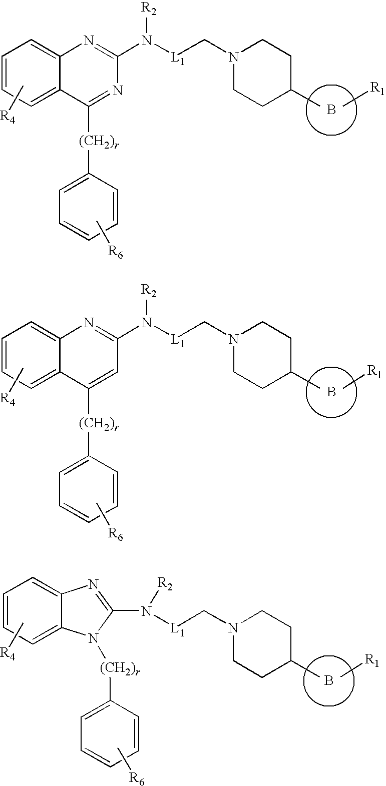

Of the permitted alternatives for R₁, hydrogen is often preferred.

Specific compounds of the invention include those of the Examples herein, particularly the compounds having the following structural formulae:

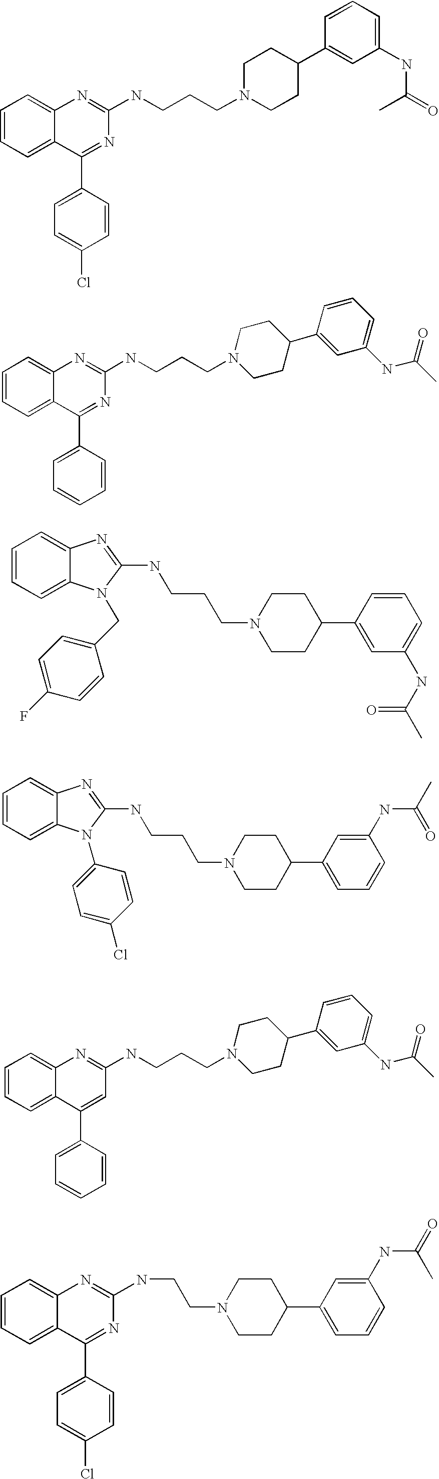

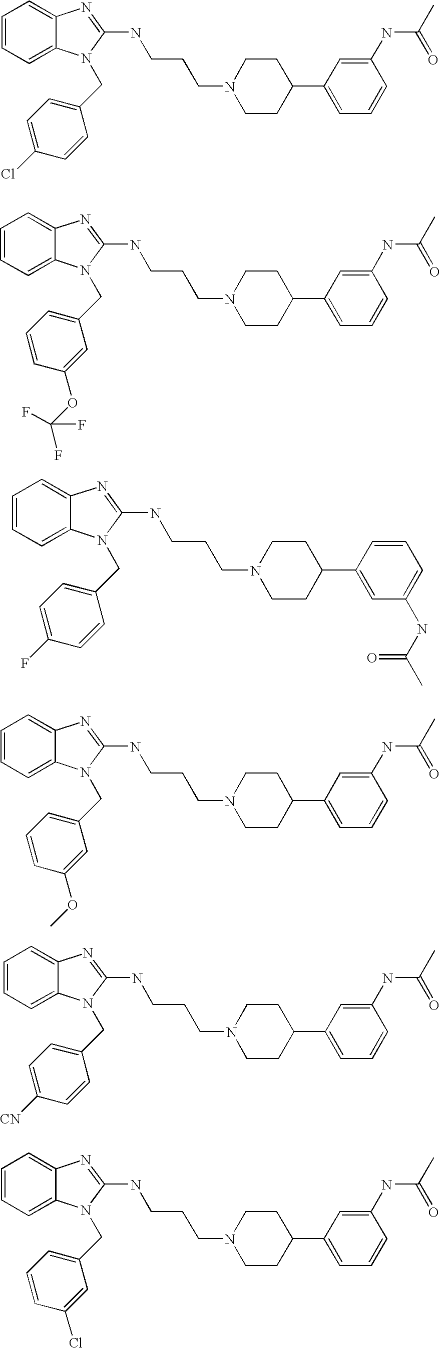

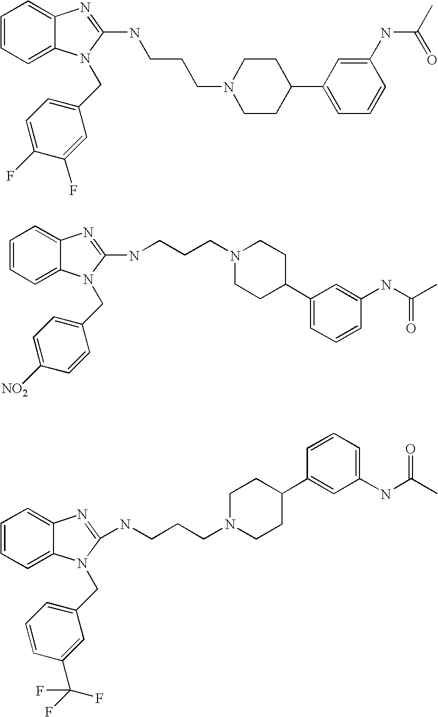

### Synthesis

There are multiple synthetic strategies for the synthesis of the compounds (I) with which the present invention is concerned, but all rely on known chemistry, known to the synthetic organic chemist. Thus, compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “Chemical Abstracts” or “Beilstein”.

For example, one strategy for the synthesis of compounds (I) involves attachment of the amino side-chain (i) to the properly functionalised ring A (ii) according to Route A or, alternatively, attachment of the functionalised group R₃ (iv) to the intermediate (iii) according to Route B as depicted in the general scheme:

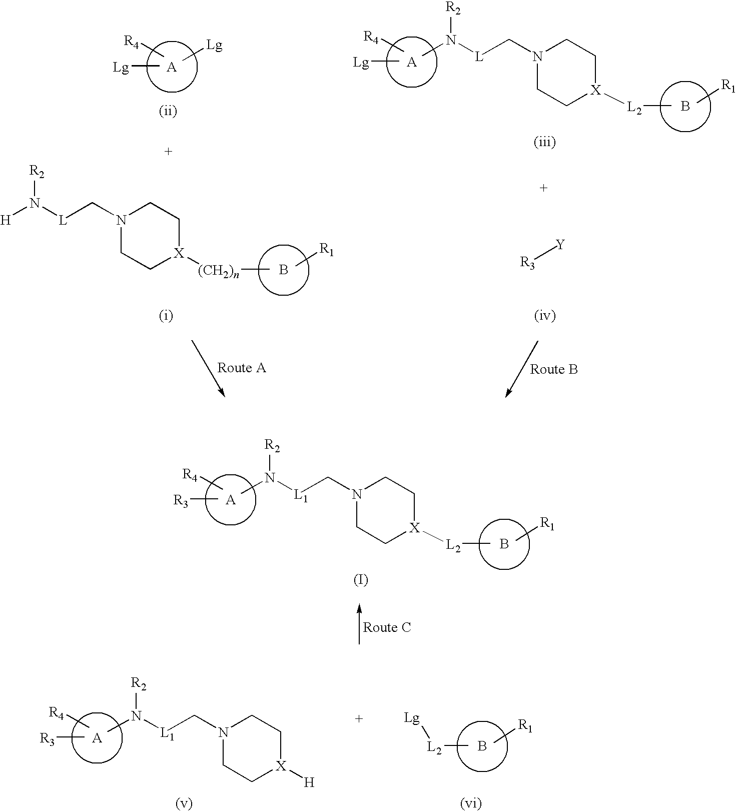

In Route A, the amine side chains (i) are reacted with the functionalised heterocyclic ring A (ii) with Lg being a leaving group like F, Cl, Br, OTs, or NO₂. The group Lg may also be a group such as hydroxyl that is converted to a leaving group in conjunction with the reaction. Alternatively, such a coupling can be promoted by transition metal catalysis such as with suitable palladium catalysts. The reactions can be facilitated by use of microwave heating.

In Route B for certain compounds, the R₃ (iv) unit is typically connected to the hetrocyclic ring A (iii) using transition metal-promoted cross-coupling reactions like Stille, Negishi and Suzuki couplings and Lg being a halide or triflate and Y being a suitable moiety like a reactive boron or tin derivative. For other compounds, the R₃ group may be attached to the A ring through nucleophilic displacement reactions.

In Route C when X is N and L₂ is not a bond, the appropriate ring B (vi) is reacted with (v) by a nucleophilic displacement with Y being for example a halide. The formation of (I) can also be done by reductive alkylation of (v) with an appropriate aldehyde (vi). When L₂ is a bond, the coupling of (v) and (vi) may be catalysed by an appropriate copper or palladium catalyst.

The various amine side chains (i)

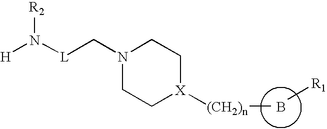

required in Route A can be prepared according known synthetic methods. For example, the specific side-chain

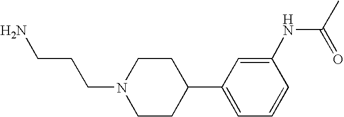

may be prepared according to a procedure described in WO 03/004027. Typically, the amine is introduced in masked form as a nitrile or azide which subsequently are reduced or carrying a general amine protecting group such as phthalimide or Boc which subsequently are deprotected. The R₁ group may be introduced at a proper point in this reaction sequence depending upon its nature and the reaction conditions. The scheme below illustrates some typical routes:

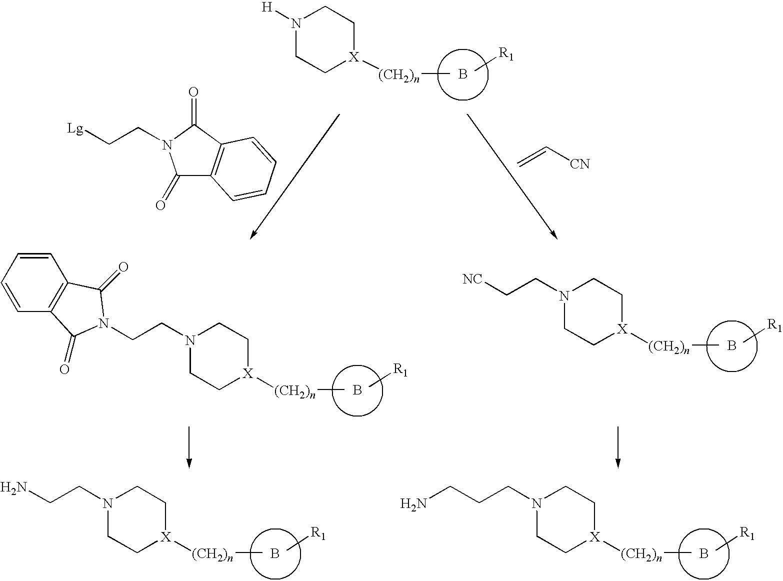

The various heterocyclic rings

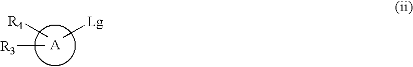

required in Routes A can be prepared according to known synthetic methods. For example, the specific quinazoline can be prepared according to the following-procedures described in literature. The synthesis is outlined below where the starting material are reacting with trichloroacetylchloride (U.S. Pat. No. 3,859,237) or ethylchloroformate (J. Med. Chem. 1987, 30, 1421) in the presence of a base followed by further reaction with ammonium acetate to give the ring closure. The chloro leaving group in the intermediate (ii) is introduced with reaction with phosphorous oxychloride.

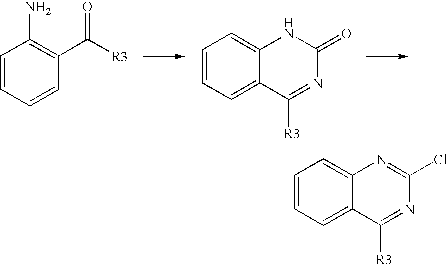

When ring A is a quinoline, the intermediate (ii) can be prepared as outlined in the scheme below according to general procedures (Justus Liebigs Ann. Chem., 1888, 245, 357-368). The condensation of the heteroaromatic ring is done by heating the starting material with PPA followed by reaction with phosphorous oxychloride to give the chlorinated intermediate.

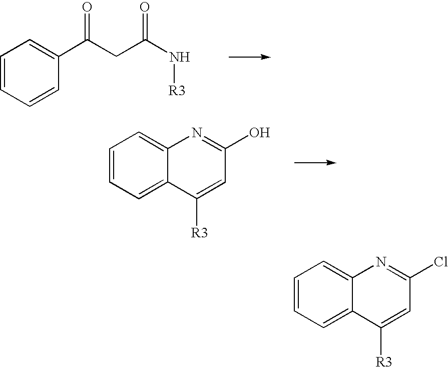

The specific benzimidazole;

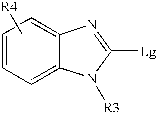

can be prepared by well known chemistry procedures. The substituted 2-amino aniline is reacted with urea by heating in an appropriate solvent (Chem. Pharm. Bull., 1993, 40, 1834-1841) to form the condensed bicyclic product which is reacted with phosphorous oxychloride giving the chloro intermediate (ii).

### Use Aspects

As mentioned above, the compounds with which the invention is concerned are capable of modulating MCH-activity. Without wishing to be bound by any theory of their mode of action in doing so, they are presently believed to bind to the MHC-1 receptor, and may in principle be antagonists, inverse agonists, agonistists or allosteres of that receptor. In the present context an agonist is defined as a compound that increases the functional activity of a MCH receptor (e.g. the signal transduction through a receptor). The term “agonist” includes partial agonist, i.e. which increases the functional activity of the receptor to a submaximal level. An inverse agonist (or negative antagonist) is defined as a compound that decreases the basal functional activity of a MCH receptor. An allosteric compound is defined as a compound that enhances or diminishes the effects of other receptor ligands. An antagonist is defined as a compound that decreases the functional activity of a MCH receptor either by inhibiting the action of an agonist or by its own intrinsic activity. Presently it is believed the activity of the present compounds, or their principal activity, is antagonistic.

Whatever their mechanism of action the compounds with which the invention is concerned are useful in the treatment of diseases or disorders (the terms “disease” and “disorder” being used interchangeably herein) which benefit from modulation of MHC activity. Examples of such diseases are referred to above, and include obesity; metabolic syndrome; Type II diabetes; bulimia; anorexia; cachexia; cardiovascular disease including dyslipidemia, myocardial infarction, and hypertension; osteoarthritis; obesity-related cancers; mental disorders such as depression, anxiety, psychosis, dementia, mood disorders, cognitive disorders, stress, memory impairment, sleep disorders, abuse disorders, delirium, sexual function disorders, reproductive function disorders; and epilepsy.

For the avoidance of doubt, references herein to “treatment” include references to curative, palliative and prophylactic treatment.

Accordingly, in another aspect, the invention provides a method of treatment of a subject suffering from a disease responsive to modulation of MCH activity, which comprised administering to the subject an amount of a compound (I) as defined and described above effective to ameliorate the disease.

In particular, compounds with which the invention is concerned are useful in the treatment of disease associated with involvement or overactivity of MCH, of which examples are given above.

In a preferred aspect, the invention provides a method of treatment of a mammalian subject suffering from obesity, metabolic syndrome, Type II diabetes, bulimia, depression, anxiety, psychosis, dementia, a mood disorder, a cognitive disorder, stress, memory impairment, an abuse disorder, or a mentally-based sexual function disorder, comprising administering to a mammal in need thereof an effective amount of a compound according to the invention.

In another preferred aspect, the invention relates to a method for modifying the feeding behaviour of a mammal, the method comprising administering to a mammal in need thereof an effective amount of a compound according to the invention.

The invention also provides to a method for the reduction of body mass, the method comprising administering to a mammal in need thereof an effective amount of a compound according to the invention.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. However, for administration to human patients, the total daily dose of the compounds of the invention may typically be in the range 1 mg to 1000 mg depending, of course, on the mode of administration. For example, oral administration may require a total daily dose of from 10 mg to 1000 mg, while an intravenous dose may only require from 1 mg to 500 mg. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly, and especially obese patients.

The compounds with which the invention is concerned may be prepared for administration by any route consistent with their pharmacokinetic properties. The orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The drug may also be formulated for inhalation, for example as a nasal spray, or dry powder or aerosol inhalers.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

The compounds with which the invention is concerned may be administered alone, or as part of a combination therapy with other drugs, especially those used for treatment of the diseases mentioned. In the treatment of obesity, a compound of the invention may be administered with an agent preventing central or peripheral food intake; modulating fat or protein metabolism or storage; preventing fat absorption; or increasing thermogenesis, In the treatment of other disorders related to obesity a compound of this invention could administered with agents used for treatment of diseases like metabolic syndrome; Type II diabetes; bulimia; cardiovascular disease including dyslipidemia, myocardial infarction, and hypertension; osteoarthritis; obesity-related cancers, In the treatment of mental disorders a compound of this invention could administered with agents used for treatment of diseases like depression, anxiety, mood disorders, abuse disorders, cognitive disorders, sleep disorders, psychosis, and dementia.

Embodiments of the invention are illustrated in the following Examples:

### General Comments

¹H-NMR data are given either in full detail or with selected characteristic peaks. LC/MS was performed on an Agilent 1100-series instrument with the column, Waters XTerra MS C18 (2.1×5 mm, 5□). The method in use; Flow: 1.0 ml/min; Gradient: 0-5 min: 10-100% MeCN, 5-7.5 min: 100% MeCN; MS-ionization mode API-ES (pos.). 5-Phenyl-1,3-dihydro-benzo[e][1,4]diazepin-2-one used in example 14 was synthesized according to a procedure described in Pharmaceutical Manufacturing Encyclopedia 2nd ed, vol. 2, p 1086.

### EXAMPLE 1

### N-[3-(1-{3-[4-(4-Chloro-phenyl)quinazolin-2-ylamino]-propyl}-piperidin-4-yl)-phenyl]-acetamide

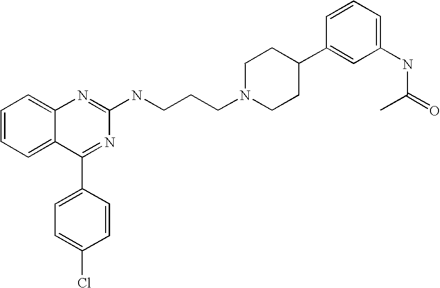

### Step 1; N-{3-[1-(2-Cyano-ethyl)-piperidin-4-yl]-phenyl}-acetamide

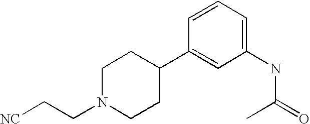

Title compound Example 1 Step 1 (4.1 g, 15.13 mmol, 94%) was synthesized according to a procedure similar to the one described in Example 10 Step 3 using acrylonitrile (2.6 ml, 40 mmol) and 4-(3-Acetylamino-phenyl)-piperidine-1-carboxylic acid tert-butyl ester (Example 12 Step 3, 5 g, 16 mmol).

¹H-NMR (300 MHz, CDCl₃); δ 1.8 (m, 4H), 2.15 (s, 3H), 3.1 (m, 2H), 6.95 (d, 1H), 7.2-7.35 (m, 3H), 7.45 (s, 1H)

### Step 2; N-{3-[1-(3-Amino-propyl)-piperidin-4-yl]-phenyl}-acetamide

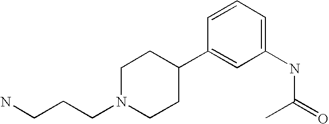

Title compound Example 1 Step 2 was synthesized in a quantitative yield according to a procedure similar to the one described in Example 10 Step 4 using Raney nickel (catalytic amount) in NH₃/methanol and N-{3-[1-(2-Cyano-ethyl)-piperidin-4-yl]-phenyl}-acetamide (Example 1 Step 1, 2.8 g, 10 mmol).

LCMS: Rt=1.55 min, m/z [MH⁺].

### Step 3

To a solution of (2-Amino-phenyl)-(4-chloro-phenyl)-methanone (4.6 g, 20 mmol), triethylamine (3.1 ml, 22 mmol) in diethylether (100 ml), which was cooled to −5° C., was trichloroacetylchloride (2.5 ml, 22 mmol) added slowly. The reaction mixture was allowed to reach room temperature and was continuously stirred until the yellow colour was disappearing whereupon water was added. The precipitate was filtered off and the organic phase was reduced giving 8 g of 2,2,2-Trichloro-N-[2-(4-chloro-benzoyl)-phenyl]-acetamide.

2,2,2-Trichloro-N-[2-(4-chloro-benzoyl)-phenyl]-acetamide (8 g, 20 mmol) was further reacted with ammonium acetate (3.2 g, 40 mmol) by heating the reactants in dmso (50 ml) to 120° C. for 2 h. The reaction mixture was cooled to room temperature then ice was added. The precipitate was collected and washed with water giving 5 g (97%) of 4-(4-chloro-phenyl)-1H-quinazolin-2-one.

4-(4-Chloro-phenyl)-1H-quinazolin-2-one (2 g, 8 mmol), phosphorous oxychloride (20 ml) and N,N-dimethylaniline (0.5 ml) were mixed and heated to 90° C. until all the reactants were dissolved. The solution was then heated 10 minutes more before cooling the reaction mixture to room temperature. The cooled mixture was portion-wise added to ice water, and neutralized with 30% NaOH before the precipitate was collected giving 6.6 g of the product 2-chloro-4-(4-chloro-phenyl)quinazoline. 2-Chloro-4-(4-chloro-phenyl)quinazoline (0.28 g, 1 mmol) and N-{3-[1-(3-Amino-propyl)-piperidin-4-yl]-phenyl}acetamide (Example 1 Step 2, 0.41 g, 1.5 mmol) were mixed and heated in the micro wave at 150° C. for 5 minutes. The reaction was extracted with dichloromethane and water and neutralized with NaOH (aq.). The crude product was purified by chromatography (silica, dichloromethane/methanol/ammoniac, 10:1:1%) giving 240 mg of the title compound Example 1.

¹H-NMR (300 MHz, CDCl₃); δ 7.18 (t, 1H), 3.02 (d, 2H), 2.50 (s, 3H).

LCMS: Rt=3.74 min, m/z [M⁺].

### EXAMPLE 2

### N-(3-{1-[3-(1-p-Tolyl-1H-benzoimidazol-2-ylamino)-propyl]-piperidin-4-yl}-phenyl)-acetamide

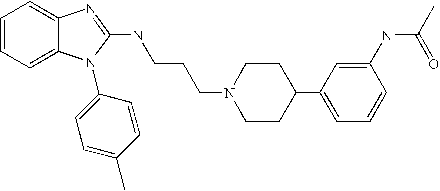

### Step 1; 1-p-Tolyl-1,3-dihydro-benzoimidazol-2-one

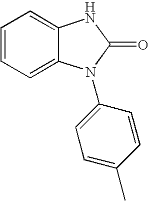

p-Tolylamine (3.2 ml, 30 mmol), 1-fluoro-2-nitro-benzene (3.2 g, 30 mmol), and triethylamine (4.2 ml, 30 mmol) were mixed and heated in the microwave at 150° C. for 1 hour and 10 minutes. The reaction mixture was stirred with water, diethylether and heptane and the formed precipitate was filtered and collected giving 4.3 g of (2-nitro-phenyl)-p-tolyl-amine.

To (2-nitro-phenyl)-p-tolyl-amine (4.1 g, 18 mmol) dissolved in ethanol (100 ml) was 10% Pd/C (catalytic amount) added. The reaction mixture was stirred under hydrogen atmosphere for 48 h, thereafter was the mixture filtered through celite and the solvent removed in vacuo yielding N-p-Tolyl-benzene-1,2-diamine. N-p-Tolyl-benzene-1,2-diamine (1.5 g, 7.5 mmol) and urea (0.6 g, 10 mmol) were dissolved in DMF (10 ml). The reaction mixture was heated to 145° C. and stirred over night. The solvent was removed in vacuo and the residue was extracted with EtOAc and water. The formed precipitate was filtered off and the filtrate was concentrated and purified by chromatography (silica, dichloromethane/methanol, 10:1) giving 0.4 g of title compound Example 1 Step 1.

LCMS: Rt=3.63 min, m/z [MH⁺].

### Step 2

1-p-Tolyl-1,3-dihydro-benzoimidazol-2-one (Example 1 Step 1, 0.4 g, 1.8 mmol) was further reacted with POCl₃ (2 ml) by heating the reactants to 110° C. for 1 h. The reaction mixture was poured on ice, neutralized with NaOH, and extracted with EtOAc. The organic phases were dried over Na₂SO₄, filtered, and evaporated, yielding 0.25 g of 2-Chloro-1-p-tolyl-1H-benzoimidazole.

The title compound Example 2 (27 mg, 0.056 mmol, 20%) was synthesized by mixing 2-Chloro-1-p-tolyl-1H-benzoimidazole (68 mg, 0.28 mmol), N-{3-[1-(3-Amino-propyl)-piperidin-4-yl]-phenyl}-acetamide (Example 1 Step 2, 130 mg, 0.47 mmol), and ethanol (5 drops) and heat the reaction mixture in the micro wave at 150° C. for 1 h and 15 minutes. The reaction was extracted with dichloromethane and water and neutralized with NaOH (aq.). The organic phases were dried over Na₂SO₄, filtered, and evaporated giving the crude product which was purified by chromatography (silica, dichloromethane/methanol/ammoniac, 10:1:1%).

¹H-NMR (300 MHz, CDCl₃); δ 7.22-7.53 (m, 8H), 7.12-7.18 (dt, 1H), 6.88-7.03 (m, 3H), 5.83 (br s, 1H), 3.67-3.73 (m, 2H), 3.00 (d, 2H), 2.52 (t, 2H), 2.37-2.50 (m, 1H), 2.22 (s, 3H), 1.73-2.01 (m, 6H), 1.23-1.32 (m, 1H).

LCMS: Rt=3.27 min, m/z [M⁺].

### EXAMPLE 3

### N-(3-{1-[3-(4-Phenyl-quinazolin-2-ylamino)-propyl]-piperidin-4-yl}-phenyl)-acetamide

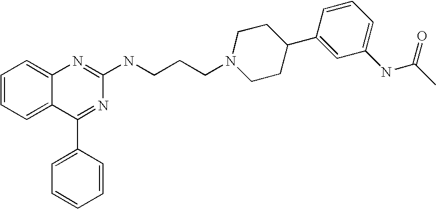

To a solution of Example 1 (100 mg, 0.20 mmol) dissolved in methanol (5 ml) were triethylamine (30 μl) and 10% Pd/C (catalytic amount) added. The reaction mixture was stirred under hydrogen atmosphere for 12 h, and was thereafter filtered through celite. The solvent was removed in vacuo and the crude product was purified by chromatography (silica, dichloromethane/methanol/ammoniac, 9:1:1%) yielding 84 mg (88%) of title compound Example 3.

LCMS: Rt=3.23 min, m/z [MH⁺].

### EXAMPLE 4

### N-[3-(1-{3-[1-(4-Fluoro-benzyl)-1H-benzoimidazol-2-ylamino]-propyl}-piperidin-4-yl)-phenyl]-acetamide

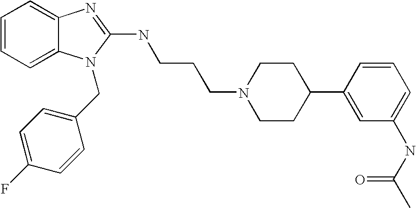

2-Chloro-1-(4-fluoro-benzyl)-1H-benzoimidazole (58 mg, 0.22 mmol) and N-{3-[1-(3-Amino-propyl)-piperidin-4-yl]-phenyl}-acetamide (Example 1 Step 2, 90 mg, 0.33 mmol) were mixed in ethanol (1 ml) and heated in the micro wave at 150° C. for 2 h. The reaction was extracted with dichloromethane and Na₂CO₃ (aq) and the combined organic phases was dried over Na₂SO₄, filtered, and evaporated giving the crude product which was purified by chromatography (silica, dichloromethane/methanol/ammoniac, 9:1:1%) giving the title compound Example 4.

¹H-NMR (300 MHz, CDCl₃); δ 7.51 (d, 1H), 7.38 (s, 1H), 7.10-7.31 (m, 6H), 6.92-7.02 (m, 4H), 6.79 (d, 1H), 6.61 (br s, 1H), 5.17 (s, 2H), 3.71 (t, 2H), 3.14 (d, 2H), 2.62 (t, 2H), 2.48-2.61 (m, 1H), 2.21 (s, 3H) 2.06-2.15 (m, 2H), 1.91-2.00 (m, 2H), 1.65-1.86 (m, 4H).

LCMS: Rt 3.48 min, m/z 499.6 [M⁺].

### EXAMPLE 5

### N-[3-(1-{3-[1-(4-Cyano-phenyl)-1H-benzoimidazol-2-ylamino]-propyl}-piperidin-4-yl)-phenyl]-acetamide

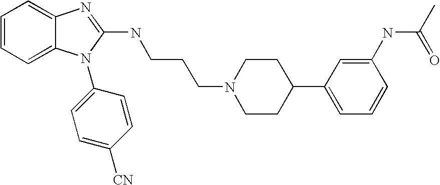

Following the same procedure as described in Example 2, 2-chloro-1-(4-cyano-phenyl)-1H-benzoimidazole (48 mg, 0.18 mmol) was synthesized and reacted with N-{3-[1-(3-Amino-propyl)-piperidin-4-yl]-phenyl}-acetamide (Example 1 Step 2, 111 mg, 0.40 mmol) giving the title compound Example 5.

¹H-NMR (300 MHz, CDCl₃); δ 7.17 (t, 1H), 6.13 (br s, 1H), 2.98 (d, 2H), 2.21 (s, 3H).

LCMS: Rt=3.09 min, m/z [M⁺].

### EXAMPLE 6

### N-[3-(1-{3-[1-(4-Chloro-phenyl)-1H-benzoimidazol-2-ylamino]-propyl}-piperidin-4-yl)-phenyl]-acetamide

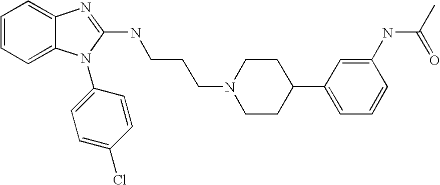

Following the same procedure as described in Example 2, 2-chloro-1-(4-chloro-phenyl)-1H-benzoimidazole (53 mg, 0.2 mmol) was synthesized and in reaction with N-{3-[1-(3-Amino-propyl)-piperidin-4-yl]-phenyl}-acetamide (Example 1 Step 2, 70 mg, 0.25 mmol) giving 51 mg (51%) of the title compound Example 6.

¹H-NMR (300 MHz, CDCl₃); δ 7.84 (s, 1H), 5.60 (s, 1H), 2.96 (d, 2H), 2.20 (s, 3H).

LCMS: Rt=1.90 min, m/z [M⁺].

### EXAMPLE 7

### N-(3-{1-[3-(4-Phenyl-quinolin-2-ylamino)-propyl]-piperidin-4-yl}-phenyl)-acetamide

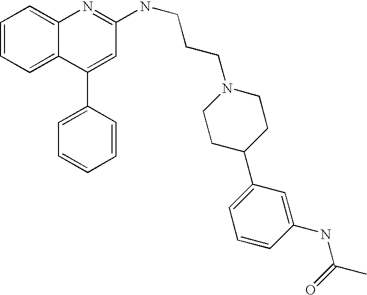

### Step 1; 4-Phenyl-quinolin-2-ol

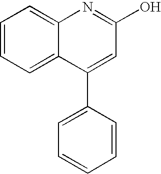

2-Benzoylacetanilide (200 mg, 0.80 mmol) were mixed with PPA (excess, solvent) and heated to 110° C. and stirred for 30 minutes. The reaction mixture was cooled and then poured on ice. The title compound Example 7 Step 1 was collected as a precipitate in quantitative yield.

LCMS: Rt=1.41 min, m/z [MH⁺].

### Step 2; 2-Chloro phenyl-quinoline

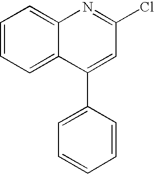

To 4-Phenyl-quinolin-2-ol (Example 7 Step 1, 150 mg, 0.7 mmol) was phosphorous oxychloride (2 ml) added. The reactants were heated to 110° C. and stirred for 30 minutes. The reaction mixture was cooled to room temperature before it was added to ice water. The water phase was neutralized with 30% NaOH and the extracted with EtOAc. The combined organic phases were dried over Na₂SO₄, filtered, and evaporated yielding 85 mg (51%) of title compound Example 7 Step 2.

LCMS: Rt=4.84 min, m/z [M⁺].

### Step 3

The title compound Example 7 (37 mg, 0.077 mmol, 22%) was synthesized by heating 2-chloro-4-phenyl-quinoline (Example 7 Step 2, 85 mg, 0.35 mmol) and N-{3-[1-(3-Amino-propyl)-piperidin-4-yl]-phenyl}-acetamide (Example 1 step 2, 150 mg, 0.50 mmol) in the micro wave at 150° C. for 20 minutes. The reaction was extracted with EtOAc and water and neutralized with NaOH (aq.). The organic phases were dried over Na₂SO₄, filtered, and evaporated giving the crude product which was purified by chromatography (silica, dichloromethane/methanol/ammoniac, 10:1:1%).

¹H-NMR (300 MHz, CDCl₃); δ 6.95 (d, 1H), 6.60 (s, 1H), 5.94 (br s, 1H), 2.17 (s, 3H).

LCMS: Rt=3.63 min, m/z [M⁺].

### EXAMPLE 8

### N-[3-(1-{3-[1-(4-Methoxy-phenyl)-1H-benzoimidazol-2-ylamino]-propyl}-piperidin-4-yl)-phenyl]-acetamide

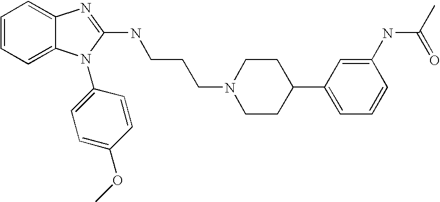

Following the same procedure as described in Example 2, 2-chloro-1-(4-methoxy-phenyl)-1H-benzoimidazole (108 mg, 0.40 mmol) was synthesized and reacted with N-{3-[1-(3-Amino-propyl)-piperidin-4-yl]-phenyl}-acetamide (Example 1 Step 2, 225 mg, 0.80 mmol) giving 60 mg (30%) of the title compound Example 8.

¹H-NMR (300 MHz, CDCl₃); δ 7.90 (s, 1H), 5.68 (s, 1H), 2.98 (d, 2H), 2.24 (s, 3H).

LCMS: Rt=3.12 min, m/z [M⁺].

### EXAMPLE 9

### N-[3-[1-{3-(3-Chloro-phenyl)-1H-benzoimidazol-2-ylamino]-propyl}-piperidin-4-yl)-phenyl]-acetamide

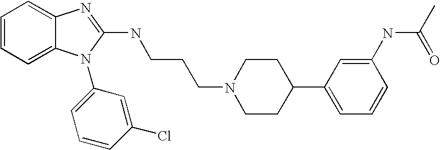

According to the procedure described in Example 2, 2-chloro-1-(3-chloro-phenyl)-1H-benzoimidazole (41 mg, 0.15 mmol) was synthesized and reacted with N-{3-[1-(3-Amino-propyl)-piperidin-4-yl]-phenyl}-acetamide (Example 1 Step 2, 100 mg, 0.36 mmol) giving the title compound Example 9.

¹H-NMR (300 MHz, CDCl₃); δ 5.83 (br s, 1H), 3.00 (d, 2H), 2.22 (s, 3H), 1.76 (d, 2H).

LCMS: Rt=3.39 min, m/z [M⁺].

### EXAMPLE 10

### 3-(4-Benzo[1,3]dioxol-5-yl-piperidin-1-yl)-propyl]-[4-(4-chloro-phenyl)-quinazolin-2-yl]-amine

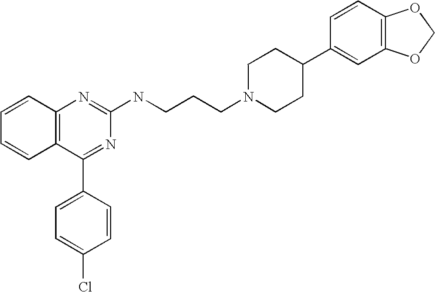

### Step 1; 4-Methanesulfonyloxy-3,6-dihydro-2H-pyridine-1-carboxylic acid tert-butyl ester

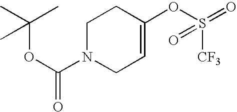

To a cooled (−10° C.) solution of diisopropylamine (2.05 ml, 14.63 mmol) in dry T.H.F (15 ml) was added, under an argon atmosphere, a 1.6M butyllithium solution in n-hexanes (9.15 ml, 14.63 mmol). After stirring for 20 min. at a temperature of −10-0° C., the mixture was cooled to −78° C. and a solution of N-Boc-4-oxo-piperidine (2.65 g, 13.30 mmol) in dry T.H.F (15 ml) was slowly added. Stirring was continued for a further 30 min. A solution of N-phenyl trifluoromethylsulfonimide (5 g, 14.0 mmol) was then added dropwise. The reaction mixture was stirred overnight and temperature was allowed to reach RT. Solvent was removed in vacuo. The crude oil was diluted with a EtOAc/Heptane mixture (1/9, 100 ml) and purified through a short alumina plug (Ø: 7 cm, H: 5 cm). The alumina plug was washed with EtOAc/Heptane (1/9, 6×200 ml). The filtrates were combined and concentrated in vacuo to give the title compound Example 10 Step 1 as an orange oil (3.79 g, 11.45 mmol, 86%).

¹H-NMR (300 MHz, CDCl₃); δ: 1.49 (s, 9H), 2.46 (m, 2H), 3.65 (t, 2H), 4.06 (d, 2H), 5.78 (s, 1H)

### Step 2; 4-Benzo[1,3]dioxol-5-yl-3,6-dihydro-2H-pyridine-1-carboxylic acid tert-butyl ester

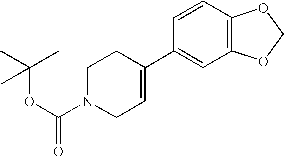

4-Methanesulfonyloxy-3,6-dihydro-2H-pyridine-1-carboxylic acid tert-butyl ester (Example 10 Step 1, 1.2 g, 3.62 mmol) and 3,4-methylenedioxybenzene boronic acid (0.69, 3.62 mmol) were mixed in a 2M K₂CO₃/D.M.E (1/1, 25 ml) biphasic solution. Nitrogen gas was bubbled through for 20 min. Bis(triphenylphosphine)PdCl^{(II)} (127 mg, 0.18 mmol) was then added and the reaction mixture was heated to 80° C. for 3 h. After cooling, stirring was continued overnight. The mixture was filtered through a celite pad. The filtrate was partitioned between EtOAc and water. The organic phase was washed with water (2×), dried over MgSO₄ and concentrated in vacuo. The residue was chromatographed over silica gel (flash, 12 g silica, eluent EtOAc/Hept.:0/100 (60 ml) then 1/90) to give the title compound Example 10 Step 2 as a colourless oil (900 mg, 2.97 mmol, 82%).

¹H-NMR (300 MHz, CDCl₃); δ: 1.50 (s, 9H), 2.50 (m, 2H), 3.63 (t, 2H), 4.06 (m, 2H), 5.93 (bs, 1H), 5.97 (s, 2H) 6.80-6.89 (m, 3H)

LCMS: Rt=3.64 min, m/z [2M+23]⁺

### Step 3; 3-(4-Benzo[1,3]dioxol-5-yl-3,6-dihydro-2H-pyridin-1-yl)-propionitrile

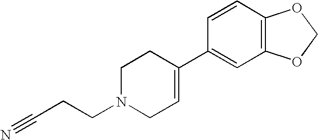

A solution of 4-Benzo[1,3]dioxol-5-yl-3,6-dihydro-2H-pyridine-1-carboxylic acid tert-butyl ester (Example 10 Step 2, 900 mg, 2.97 mmol) in a 10% T.F.A/CH₂Cl₂ (20 ml) mixture was stirred at RT for 1 h. solvent was removed in vacuo. The residue was taken up in methanol (20 ml) and solid NaHCO₃ was added until pH=8. The mixture was cooled to 0° C. and acrylonitrile (0.49 ml, 7.42 mmol) was added dropwise. After completion, the reaction mixture was allowed to stir at RT overnight. EtOAc (20 ml) and water (40 ml) were added and stirring was continued for a further 1 h. The phases were separated and the water phase was extracted with EtOAc. The organic phases were combined, dried over MgSO₄ and concentrated in vacuo. The residue was purified over silica gel chromatography (flash, 12 g silica, eluent EtOAc/Hept.: 5/95 to 4/1 in 30 min.) to give the title compound Example 10 Step 3 as a semi solid (470 mg, 1.83 mmol, 76%).

LCMS: Rt=3.57 min, m/z [MH]⁺

### Step 4; 3-(4-Benzo[1,3]dioxol-5-yl-piperidin-1-yl)-propylamine

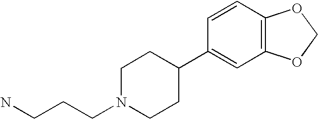

A mixture of 3-(4-Benzo[1,3]dioxol-5-yl-3,6-dihydro-2H-pyridin-1-yl)-propionitrile (Example 10 Step 3, 470 mg, 1.83 mmol) and Raney nickel (catalytic amount) in a NH₃/Methanol solution (50 ml) was stirred at 30° C. for 18 h under a hydrogen atmosphere. The catalyst was filtered off and the filtrate concentrated in vacuo. The residue was dissolved in methanol (50 ml) and 10% Pd/C (catalytic amount) was added. The reaction mixture was stirred at 30° C. for 18 h under a hydrogen atmosphere. The catalyst was filtered off and the filtrate concentrated in vacuo to give the title compound Example 10 Step 4 as an oil (390 mg, 1.49 mmol, 81%).

LCMS: Rt=3.06 min, m/z [MH]⁺

### Step 5;

Title compound Example 10 (22.4 mg, 0.045 mmol, 18%) was synthesized according to a procedure similar to the one described in Example 1 Step 3 using 2-Chloro-4-(4-chloro-phenyl)quinazoline (Example 1 Step 3, 69.2 mg, 0.25 mmol) and 3-(4-Benzo[1,3]dioxol-5-yl-piperidin-1-yl)-propylamine (Example 10 Step 4, 66 mg, 0.25 mmol).

LCMS: Rt=5.47 min, m/z [M]⁺

### EXAMPLE 11

### 3-(1-{3-[4-(4-Chloro-phenyl)quinazolin-2-ylamino]-propyl}-piperidin-4-yl)-N-methyl-benzamide

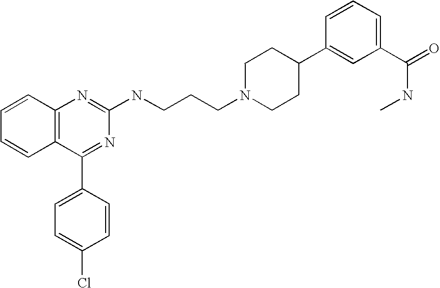

### Step 1; 4-(3-Methylcarbamoyl-phenyl)-3,6-dihydro-2H-pyridine-1-carboxylic acid tert-butyl ester

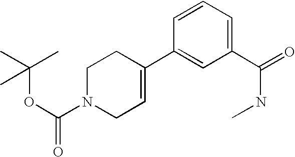

Title compound Example 11 Step 1 (680 mg, 2.15 mmol, 53%) was synthesized according to a procedure similar to the one described in Example 10 Step 2 using 4-methanesulfonyloxy-3,6-dihydro-2H-pyridine-1-carboxylic acid tert-butyl ester (1.35 g, 4.08 mmol) and 3-(N-methylaminocarbonyl)phenyl boronic acid (730 mg, 4.08 mmol).

LCMS: Rt=3.83 min, m/z [M+23]⁺

### Step 2; 4-(3-Methylcarbamoyl-phenyl)-piperidine-1-carboxylic acid tert-butyl ester

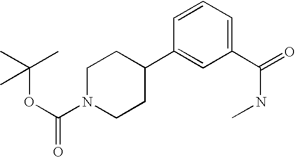

4-(3-Methylcarbamoyl-phenyl)-3,6-dihydro-2H-pyridine-1-carboxylic acid tert-butyl ester (Example 11 Step 1, 670 mg, 2.12 mmol) and 10% Pd/C (catalytic amount) were stirred in methanol (50 ml) at 35° C. for 1 h under a hydrogen atmosphere. The catalyst was filtered off and the filtrate was concentrated in vacuo to give the title compound Example 11 Step 2 as a colourless gum (670 mg, 2.10 mmol, 99%).

LCMS: Rt=3.70 min, m/z [M+23]⁺

### Step 3; 3-[1-(2-Cyano-ethyl)-piperidin-4-yl]-N-methyl-benzamide

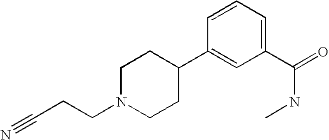

Title compound Example 11 Step 3 (104 mg, 0.38 mmol, 18%) was synthesized according to a procedure similar to the one described in Example 10 Step 3 using acrylonitrile (0.35 ml, 5.26 mmol) and 4-(3-Methylcarbamoyl-phenyl)-piperidine-1-carboxylic acid tert-butyl ester (Example 11 Step 2, 670 mg, 2.10 mmol).

LCMS: Rt=3.83 min, m/z [M+23]⁺

### Step 4; 3-[1-(3-Amino-propyl)-piperidin-4-yl]-N-methyl-benzamide

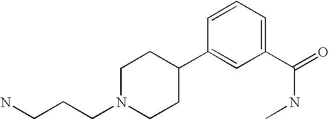

A mixture of 3-[1-(2-Cyano-ethyl)-piperidin-4-yl]-N-methyl-benzamide (Example 11 Step 3, 104 mg, 0.38 mmol) and Raney nickel (catalytic amount) in NH₃/Methanol solution (20 ml) was stirred at RT for 18 h under a hydrogen atmosphere. The catalyst was filtered off and the filtrate concentrated in vacuo to give title compound Example 11 Step 4 as a pale yellow liquid (105 mg, 0.38, 100%).

LCMS: Rt=broad peak, m/z [MH]⁺

### Step 5;

Title compound Example 11 (35 mg, 0.068 mmol, 18%) was synthesized according to a procedure similar to the one described in Example 1 Step 3 using 2-Chloro-4-(4-chloro-phenyl)quinazoline (Example 1 Step 3, 105 mg, 0.38 mmol) and 3-[1-(3-Amino-propyl)-piperidin-4-yl]-N-methyl-benzamide (Example 11 Step 4, 105 mg, 0.38 mmol).

LCMS: Rt=3.60 min, m/z [M]⁺

### EXAMPLE 12

### N-[3-(1-{2-[4-(4-Chloro-phenyl)quinazolin-2-ylamino]-ethyl}-piperidin-4-yl)-phenyl]-acetamide

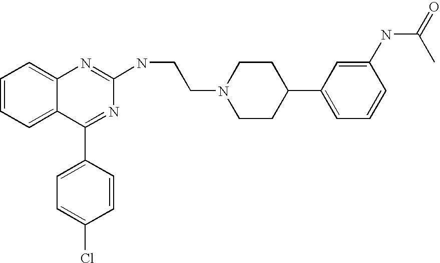

### Step 1; 4-(3-Nitro-phenyl)-3,6-dihydro-2H-pyridine-1-carboxylic acid tert-butyl ester

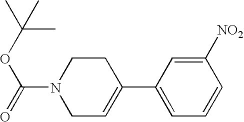

Title compound Example 12 Step 1 (950 mg, 3.12 mmol, 86%) was synthesized according to a procedure similar to the one described in Example 10 Step 2 using 4-methanesulfonyloxy-3,6-dihydro-2H-pyridine-1-carboxylic acid tert-butyl ester (1.2 g, 3.62 mmol) and 3-nitrophenyl boronic acid (600 mg, 3.62 mmol).

¹H-NMR (300 MHz, CDCl₃); δ: 1.52 (s, 9H), 2.57 (bs, 2H), 3.69 (t, 2H), 4.15 (bs, 2H), 6.21 (bs, 1H), 7.52 (t, 1H), 7.69 (d, 1H), 8.1 (d, 1H), 8.24 (s, 1H)

LCMS: Rt=3.79 min, m/z [M+23]⁺

### Step 2; 4-(3-Amino-phenyl)-piperidine-1-carboxylic acid tert-butyl ester

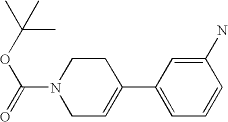

A mixture of 4-(3-Nitro-phenyl)-3,6-dihydro-2H-pyridine-1-carboxylic acid tert-butyl ester (Example 12 Step 1, 950 mg, 3.12 mmol) and 10% Pd/C (catalytic amount) in methanol (20 ml) was stirred at 35° C. for 3 h under a hydrogen atmosphere. The catalyst was filtered off and the filtrate was concentrated in vacuo to give a grey solid. Small amounts of diethyl ether (2×5 ml) were added to the solid. After decantation and removal of the solvent, the solid was dried in vacuo to give title compound Example 12 Step 2 as a white solid (800 mg, 2.90 mmol, 86%).

LCMS: Rt=2.49 min, m/z [M+23]⁺

### Step 3; 4-(3-Acetylamino-phenyl)-piperidine-1-carboxylic acid tert-butyl ester

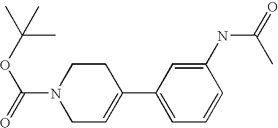

To a cooled (0° C.) solution of 4-(3-Amino-phenyl)-piperidine-1-carboxylic acid tert-butyl ester (Example 12 Step 2, 800 mg, 2.90 mmol) in CH₂Cl₂ was slowly added acetic anhydride (0.41 ml, 4.35 mmol). After completion, the reaction mixture was stirred overnight and allowed to slowly reach RT. Saturated aqueous NaHCO₃ (10 ml) was added and the mixture was stirred vigorously for 3 h at RT. The phases were separated. The aqueous phase was extracted with CH₂Cl₂. The organic phases were combined, dried over MgSO₄ and concentrated in vacuo to give title compound Example 12 Step 3 as an oil which crystallized upon standing (900 mg, 2.82, 97%).

¹H-NMR (300 MHz, CDCl₃); □: 1.50 (s, 9H), 2.18 (s, 3H), 6.95 (d, 1H), 7.28 (m, 3H), 7.42 (bs, 1H)

LCMS: Rt=3.71 min, m/z [M+23]⁺

### Step 4; N-(3-Piperidin-4-yl-phenyl)-acetamide

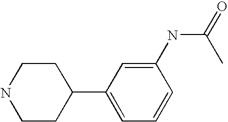

A solution of 4-(3-Acetylamino-phenyl)-piperidine-1-carboxylic acid tert-butyl ester (Example 12 Step 3, 454 mg, 1.43 mmol) in a 10% T.F.A/CH₂Cl₂ (30 ml) mixture was stirred at RT for 1.5 h. Solvent was removed in vacuo. The residue was taken up in ethylacetate and 1M aqueous NaOH was added until pH>12. Solid NaCl was then added to the aqueous phase and extraction was carried out with ethyl acetate. The organic phase was dried over MgSO₄ and concentrated in vacuo to give title compound Example 12 Step 4 as a solid (280 mg, 1.28 mmol, 90%)

LCMS: Rt=0.52 min, m/z [MH]⁺

### Step 5; N-(3-{1-[2-(1,3-Dioxo-1,3-dihydro-isoindol-2-yl)-ethyl]-piperidin-4-yl}-phenyl)-acetamide

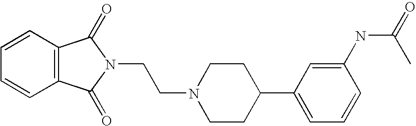

To a solution of N-(2-bromoethyl) phtalamide (130 mg, 0.51 mmol) and N-(3-Piperidin-4-yl-phenyl)-acetamide (Example 12 Step 4, 100 mg, 0.46 mmol) in acetonitrile (6 ml) was added solid K₂CO₃ (130 mg, 0.92 mmol). The reaction mixture was refluxed for 2 h. After cooling, water was added and the mixture was extracted with CH₂Cl₂. The organic phase was dried over MgSO₄ and concentrated in vacuo. The residue was purified over silica gel chromatography (eluent: CH₂Cl₂/MeOH/NH₃: 100/0/0 then 94/5/1) to give title compound Example 12 Step 5 (140 mg, 0.36 mmol, 78%).

LCMS: Rt=2.43 min, m/z [MH]⁺

### Step 6; N-{3-[1-(2-Amino-ethyl)-piperidin-4-yl]-phenyl}-acetamide

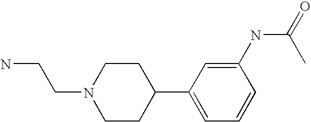

A solution of N-(3-{1-[2-(1,3-Dioxo-1,3-dihydro-isoindol-2-yl)-ethyl]-piperidin-4-yl}-phenyl)-acetamide (Example 12 Step 5, 140 mg, 0.36 mmol) in 33% methylamine/ethanol (5 ml) was stirred at RT overnight. Solvent was removed in vacuo. The residue was purified over a SCX column to remove side non basic products to give title compound Example 12 Step 6 (90 mg, 0.34 mmol, 94%, crude yield) which was contaminated with Example 12 Step 5 starting material. The title compound was used without further purification in Step 7.

LCMS: Rt=1.38 min, m/z [MH]⁺

### Step 7;

Title compound Example 12 (27 mg, 0.054 mmol, 28%) was synthesized according to a procedure similar to the one described in Example 1 Step 3 using 2-Chloro-4-(4-chloro-phenyl)quinazoline (Example 1 Step 3, 53 mg, 0.19 mmol) and N-{3-[1-(2-Amino-ethyl)-piperidin-4-yl]-phenyl}-acetamide (Example 12 Step 6, 50 mg, 0.19 mmol) in ethanol (2 ml).

LCMS: Rt=3.90 min, m/z [M]+

### EXAMPLE 13

### N-[3-(1-{1-[4-(4-Chloro-phenyl)quinazolin-2-yl]-azetidin-3-ylmethyl}-piperidin-4-yl)-phenyl]-acetamide

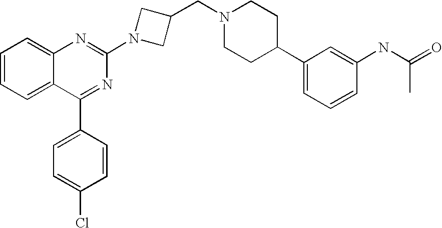

### Step 1; 3-[4-(3-Acetylamino-phenyl)-piperidin-1-ylmethyl]-azetidine-1-carboxylic acid tert-butyl ester

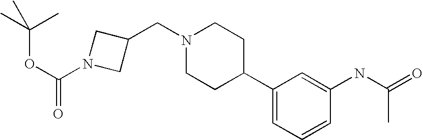

To a cooled (0° C.) solution of 3-Formyl-azetidine-1-carboxylic acid tert-butyl ester (20 mg, 0.11 mmol) and N-(3-Piperidin-4-yl-phenyl)-acetamide (Example 12 Step 4, 20 mg, 0.09 mmol) in methanol (2 ml) was added NaBH₄ (10 mg, 0.16 mmol). The pH of the solution was adjusted to 6 by addition of acetic acid and the reaction mixture was then stirred at RT for 4 h. Ethyl acetate and 4M aqueous NaOH were added. The organic phase was separated, dried over MgSO₄ and concentrated in vacuo to give title compound Example 13 Step 1 (36.6 mg, 0.09 mmol, 100% crude yield).

LCMS: Rt=2.70 min, m/z [MH]⁺

### Step 2

A solution of 3-[4-(3-Acetylamino-phenyl)-piperidin-1-ylmethyl]-azetidine-1-carboxylic acid tert-butyl ester (Example 13 Step 1, 36.6 mg, 0.09 mmol) in a 10% T.F.A/CH₂Cl₂ (2 ml) mixture was stirred at RT for 1 h. Solvent was removed in vacuo. The residue was taken up in ethylacetate and 1M aqueous NaOH was added until pH>12. Solid NaCl was then added to the aqueous phase and extraction was carried out with ethyl acetate. The organic phase was dried over MgSO₄ and concentrated in vacuo. The residue was reacted in ethanol (2 ml) with 2-Chloro-4-(4-chloro-phenyl)-quinazoline (Example 1 Step 3, 30 mg, 0.11 mmol) according to the procedure described in Example 1 Step 3 to give, after usual work-up, title compound Example 13 (9 mg, 0.017 mmol, 19%).

LCMS: Rt=4.13 min, m/z [M]⁺

### EXAMPLE 14

### N-(3-{1-[3-(5-Phenyl-3H-benzo[e][1,4]diazepin-2-ylamino)-propyl]-piperidin-4-yl}-phenyl)-acetamide

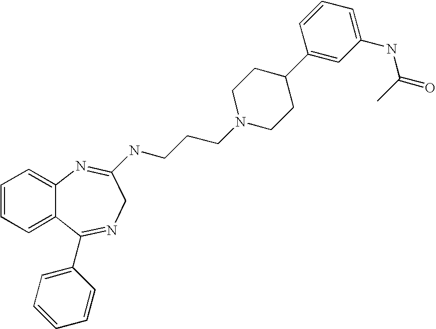

The title compound Example 14 was synthesized by first reacting 5-Phenyl-1,3-dihydro-benzo[e][1,4]diazepin-2-one (120 mg, 0.50 mmol) with sodium hydride (50%) (25 mg, 0.60 mmol) in dry DMF (5 ml) at room temperature, a precipitate was immediately formed. After stirring at room temperature for 20 minutes diethylchloro phosphate (80 □l, 0.60 mmol) was added. The precipitate came into solution and the reaction was stirred for 30 minutes before N-{3-[1-(3-Amino-propyl)-piperidin-4-yl]-phenyl}-acetamide (Example 1 Step 2, 150 mg, 0.60 mmol) was added. The reaction mixture was then heated to 70° C. overnight, cooled to room temperature, and extracted with water/EtOAc. The combined organic phases were dried over Na₂SO₄, filtered, and evaporated giving the crude product which was purified by chromatography (silica, dichloromethane/methanol/ammoniac, 10:1:1%) giving 36 mg (15% in over all yield) of N-(3-{1-[3-(5-Phenyl-3H-benzo[e][1,4]diazepin-2-ylamino)-propyl]-piperidin-4-yl}-phenyl)-acetamide.

¹H-NMR (300 MHz, CDCl₃); δ 8.54 (s, 1H), 6.94 (t, 1H), 6.87 (d, 1H), and 2.17 (s, 3H). LCMS: Rt=3.06 min, m/z [M⁺].

### EXAMPLE 15

### N-(3-{1-[3-(4-Phenyl-pyrimidin-2-ylamino)-propyl]-piperidin-4-yl}-phenyl)-acetamide

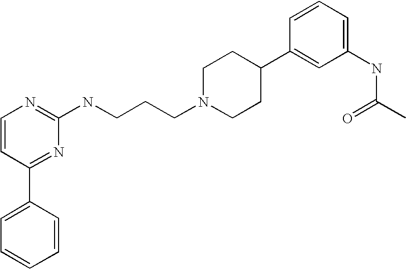

To 2-Chlorophenyl-pyrimidine (95 mg, 0.50 mmol) dissolved in ethanol (0.5 ml) was N-{3-[1-(3-Amino-propyl)-piperidin-4-yl]-phenyl}-acetamide (Example 1 Step 2, 150 mg, 0.55 mmol) added and the reaction mixture was heated in the micro wave at 150° C. for 10 minutes. The reaction was extracted with EtOAc and water and the combined organic phases were dried over Na₂SO₄, filtered, and evaporated giving the crude product which was purified by chromatography (silica, dichloromethane/methanol, 10:1) giving 40 mg (19%) of title compound Example 15.

¹H-NMR (300 MHz, CDCl₃); δ 6.90 (d, 1H), 7.23 (t, 1H) and 2.03 (s, 3H).

LCMS: Rt=0.58 min, m/z [M⁺].

### EXAMPLE 16

### N-{3-[1-(3-{4-[(Pyridin-2-ylmethyl)amino]-quinazolin-2-ylamino}-propyl)-piperidin 4-yl]-phenyl}-acetamide

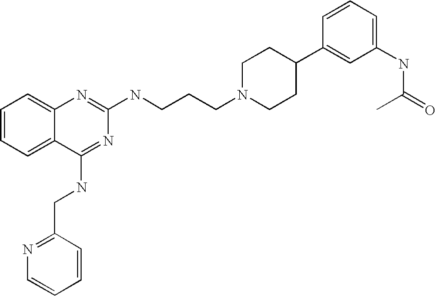

To a solution of 2,4-dichloroquinazoline (100 mg, 0.50 mmol) dissolved in diethylether (2 ml) was 2-(aminomethyl)pyridine (52 μl, 0.50 mmol) added and the reaction mixture was stirred at room temperature for 12 h. The formed precipitate was collected giving 77 mg (57%) (2-Chloro-quinazolin-4-yl)-pyridin-2-ylmethyl-amine. LCMS: Rt=2.88 min, m/z [M⁺].

To (2-Chloro-quinazolin-4-yl)-pyridin-2-ylmethyl-amine (48 mg, 0.18 mmol) dissolved in ethanol (1 ml) was N-{3-[1-(3-Amino-propyl)-piperidin-4-yl]-phenyl}-acetamide (Example 1 Step 2, 75 mg, 0.27 mmol) added and the reaction mixture was heated in the micro wave at 150° C. for 15 minutes. The reaction was extracted with EtOAc and water and the combined organic phases was dried over Na₂SO₄, filtered, and evaporated giving the crude product which was purified by chromatography (silica, dichloromethane/methanol/ammoniac, 9:1:1%) giving 27 mg of title compound Example 16.

¹H-NMR (300 MHz, CDCl₃); δ 8.6 (d, 1H), 7.12 (t, 1H), 4.95 (d, 2H), and 1.95 (s, 3H).

LCMS: Rt=2.70 min, m/z [M⁺].

### EXAMPLE 17

### N-[3-(1-{3-[4-(2-Pyrrolidin-1-yl-ethylamino)-quinazolin-2-ylamino]-propyl}-piperidin-4-yl)-phenyl]-acetamide

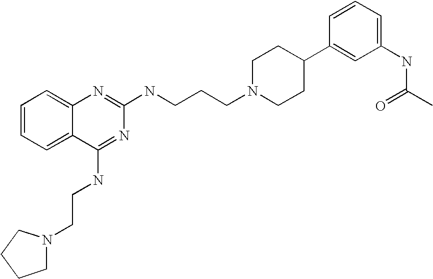

To a solution of 2,4-dichloroquinazoline (100 mg, 0.50 mmol) dissolved in diethylether (2 ml) was 2-pyrrolidin-1-yl-ethylamine (63 μl, 0.50 mmol) added and the reaction mixture was stirred at room temperature for 12 h. The formed precipitate was collected giving 137 mg (98%) (2-chloro-quinazolin-4-yl)-(2-pyrrolidin-1-yl-ethyl)-amine. LCMS: Rt=1.80 min, m/z [M⁺].

To (2-chloro-quinazolin-4-yl)-(2-pyrrolidin-1-yl-ethyl)-amine (52 mg, 0.19 mmol) dissolved in ethanol (1 ml) was N-{3-[1-(3-Amino-propyl)-piperidin-4-yl]-phenyl}-acetamide (Example 1 Step 2, 61 mg, 0.22 mmol) added and the reaction mixture was heated in the micro wave at 150° C. for 15 minutes. The reaction was extracted with EtOAc and Na₂CO₃ (aq) and the combined organic phases was dried over Na₂SO₄, filtered, and evaporated giving the crude product which was purified by chromatography twice (silica, dichloromethane/methanol/ammoniac, 9:1:1%) giving 29 mg (29%) of title compound Example 17.

¹H-NMR (300 MHz, CDCl₃); δ 7.83 (s, 1H), 7.45-7.61 (m, 4H), 7.34 (s, 1H), 7.24 (t, 1H), 7.08 (t, 1H), 6.6 (br s, 1H), 6.0 (br s, 1H), 3.68 (t, 2H), 3.59 (t, 2H), 3.07 (d, 2H), 2.79 (t, 4H), 2.55-2.71 (m, 4H), 2.51 (t, 4H), 2.18 (s, 3H), 1.98-2.08 (m, 2H), 1.76-1.87 (m, 8H).

LCMS: Rt=5.32 and 6.22 min, m/z [M⁺] (Two broad product peaks).

### EXAMPLE 18

### N-[3-(1-{3-[4-(2-Morpholino-4-yl-ethylamino)-quinazolin-2-ylamino]-propyl}-piperidin-4-yl)-phenyl]-acetamide

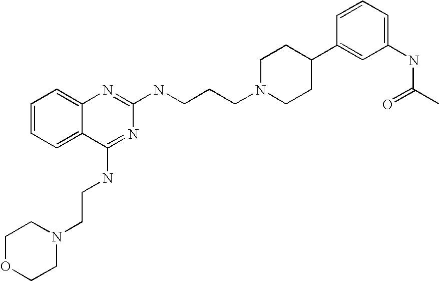

To a solution of 2,4-dichloroquinazoline (100 mg, 0.50 mmol) dissolved in diethylether (2 ml) was 2-morpholin-4-yl-ethylamine (0.50 mmol) added and the reaction mixture was stirred at room temperature for 12 h. The reaction was extracted with diethylether and NaOH (aq) and the combined organic phases were dried over Na₂SO₄, filtered, and evaporated giving 70 mg (48%) of (2-Chloro-quinazolin-4-yl)-(2-morpholin-4-yl-ethyl)-amine. LCMS: Rt=2.27 min, m/z [MH⁺]. To (2-Chloro-quinazolin-4-yl)-(2-morpholin-4-yl-ethyl)-amine (70 mg, 0.24 mmol) dissolved in ethanol (2 ml) was N-{3-[1-(3-Amino-propyl)-piperidin-4-yl]-phenyl}-acetamide (Example 1 Step 2, 72 mg, 0.26 mmol) added and the reaction mixture was heated in the micro wave at 150° C. for 15 minutes. The reaction was extracted with EtOAc and Na₂CO₃ (aq) and the combined organic phase was dried over Na₂SO₄, filtered, and evaporated giving the crude product which was purified by chromatography (silica, dichloromethane/methanol/ammoniac, 9:1:1%) giving 16 mg (13%) of title compound Example 18.

¹H-NMR (300 MHz); δ 6.37 (br s, 1H), 5.72 (br s, 1H) 3.08 (d, 2H), 2.19 (s, 3H).

LCMS: Rt=5.45 (broad) min, m/z [M⁺].

### EXAMPLE 19

### N-(3-{1-[3-(4-Morpholin-4-yl-quinazolin-2-ylamino)-propyl]-piperidin-4-yl}-phenyl)-acetamide

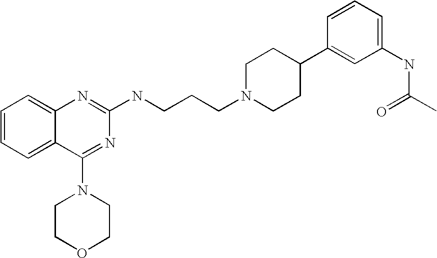

To a solution of 2,4-dichloroquinazoline (100 mg, 0.50 mmol) dissolved in diethylether (2 ml) was morpholine (44 μl, 0.50 mmol) added and the reaction mixture was stirred at room temperature for 12 h. The formed precipitate was filtered off and the filtrate evaporated giving 58 mg (46%) 2-Chloro-4-morpholin-4-yl-quinazoline. LCMS: Rt=2.59 min, m/z 249.7 [M⁺].

2-Chloro-4-morpholin-4-yl-quinazoline (100 mg, 0.40 mmol) and N-{3-[1-(3-Amino-propyl)-piperidin-4-yl]-phenyl}-acetamide (Example 1 Step 2, 115 mg, 0.42 mmol) were mixed in ethanol (1 ml) and heated in the micro wave at 150° C. for 15 minutes. The reaction was extracted with EtOAc and Na₂CO₃ (aq.) and the combined organic phases were dried over Na₂SO₄, filtered, and evaporated giving the crude product which was purified by chromatography (silica, dichloromethane/methanol/ammoniac, 100:0:0-8:2:1%) giving title compound Example 19.

¹H-NMR (300 MHz, CDCl₃); δ 7.70 (d, 1H), 6.99 (d, 1H), 3.72 (br s, 2H), 2.19 (s, 3H).

LCMS: Rt=2.98, m/z [M⁺].

### EXAMPLE 20

### (4-{3-[4-(4-Chloro-phenyl)-quinazolin-2-ylamino]-propyl}-piperazin-1-yl)-(3-trifluoromethoxy-phenyl)-methanone

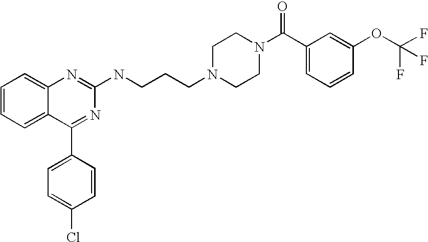

### Step 1; 4-(2-Cyano-ethyl)-piperazine-1-carboxylic acid tert-butyl ester

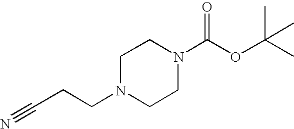

A mixture of 1-Boc-piperazine (2 g, 10.74 mmol) and acrylonitrile (0.7 ml, 10.74 mmol) was refluxed overnight under argon. The reaction mixture was cooled to RT to give title compound Example 20 Step 1 (2.56 g, 10.69 mmol, 99%) which was used in step 2 without further purification.

¹H-NMR (300 MHz, CDCl₃); δ 1.47 (s, 9H), 2.49 (m, 6H), 3.43 (m, 4H), 4.84 (s, 2H).

### Step 2; 4-(3-Amino-propyl)-piperazine-1-carboxylic acid tert-butyl ester

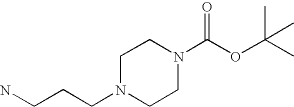

Title compound Example 20 Step 2 was synthesized in a quantitative yield according to a procedure similar to the one described in Example 10 Step 4 using Raney nickel (catalytic amount) in NH₃/methanol and 4-(2-Cyano-ethyl)-piperazine-1-carboxylic acid tert-butyl ester (Example 20 Step 1, 700 mg, 2.92 mmol).

LCMS: Rt=1.45 min (MS signal), m/z [MH⁺].

### Step 3; 4-{3-[4-(4-Chloro-phenyl)-quinazolin-2-ylamino]-propyl}-piperazine-1-carboxylic acid tert-butyl ester

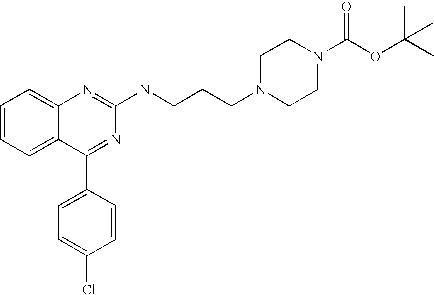

Title compound Example 20 Step 3 (1.06 g, 2.20 mmol, 73%) was synthesized according to a procedure similar to the one described in Example 1 Step 3 using 2-Chloro-4-(4-chloro-phenyl)quinazoline (Example 1 Step 3, 911 mg, 3.31 mmol) and 4-(3-Amino-propyl)-piperazine-1-carboxylic acid tert-butyl ester (Example 20 Step 2, 733 mg, 3.01 mmol) in ethanol (10 ml). The reaction mixture was heated for 40 min in the micro wave at a fixed temperature of 100° C. to avoid removal of the Boc group.

LCMS: Rt=4.56 min, m/z [M]⁺

### Step 4; [4-(4-Chloro-phenyl)quinazolin-2-yl]-(3-piperazin-1-yl-propyl)-amine

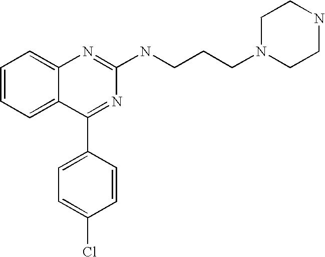

A solution of 4-{3-[4-(4-Chloro-phenyl)-quinazolin-2-ylamino]-propyl}-piperazine-1-carboxylic acid tert-butyl ester (Example 20 Step 3, 1.0 g, 2.07 mmol) in a 10% T.F.A/CH₂Cl₂ (20 ml) mixture was stirred at RT for 2 h. Solvent was removed in vacuo. The residue was taken up in CH₂Cl₂ and 1M aqueous NaOH was added until pH>12. Solid NaCl was then added to the aqueous phase and extraction was carried out with CH₂Cl₂. The organic phase was dried over MgSO₄ and concentrated in vacuo to give title compound Example 20 Step 4 (700 mg, 1.83 mmol, 88%)

LCMS: Rt=3.45 min, m/z [M]⁺

### Step 5

To a solution of [4-(4-Chloro-phenyl)-quinazolin-2-yl]-(3-piperazin-1-yl-propyl)-amine (Example 20 Step 4, 20.2 mg, 0.053 mmol) and triethylamine (excess) in dry CH₂Cl₂ was added 3-trifluoromethoxybenzoyl chloride (13 mg, 0.058 mmol). After stirring for 2 h at RT, the mixture was purified over silica gel chromatography to give title compound Example 20 (16 mg, 0.028 mmol, 53%).

LCMS: Rt=4.84 min, m/z [M]⁺

### EXAMPLE 21

### (4-{3-[4-(4-Chloro-phenyl)quinazolin-2-ylamino]-propyl}-piperazin-1-yl)-(3-methoxy-phenyl)-methanone

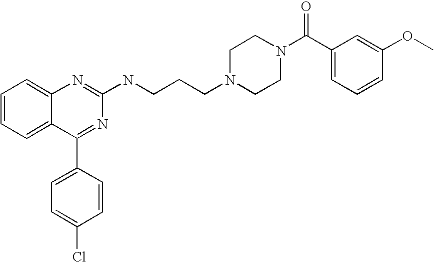

Title compound Example 21 (16 mg, 0.031 mmol, 58%) was synthesized according to a procedure similar to the one described in Example 20 Step 5 using [4-(4-Chloro-phenyl)-quinazolin-2-yl]-(3-piperazin-1-yl-propyl)-amine (Example 20 Step 4, 20.2 mg, 0.053 mmol) and 3-methoxybenzoyl chloride (10 mg, 0.058 mmol).

LCMS: Rt=4.61 min, m/z [M]⁺

### EXAMPLE 22

### 3-(4-{3-[4-(4-Chloro-phenyl)-quinazolin-2-ylamino]-propyl}-piperazine-1-carbonyl)-benzonitrile

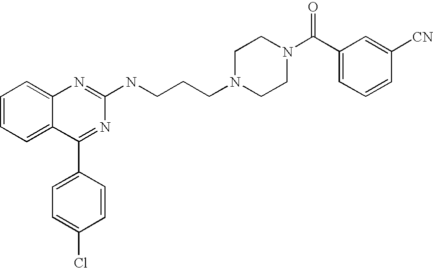

Title compound Example 22 (17 mg, 0.033 mmol, 61%) was synthesized according to a procedure similar to the one described in Example 20 Step 5 using [4-(4-Chloro-phenyl)quinazolin-2-yl]-(3-piperazin-1-yl-propyl)-amine (Example 20 Step 4, 20.6 mg, 0.054 mmol) and 3-cyanobenzoyl chloride (10 mg, 0.060 mmol).

LCMS: Rt=4.51 min, m/z [M]⁺

### EXAMPLE 23

### N-[3-(1-{3-[1-(4-Chloro-benzyl)-1H-benzoimidazol-2-ylamino]-propyl}-piperidin-4-yl)-phenyl]-acetamide

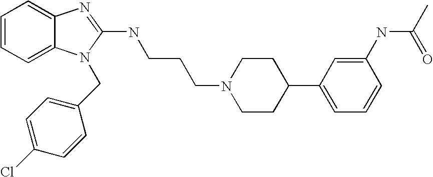

### Step 1; 2-Chloro-1-(4-chloro-benzyl)-1H-benzoimidazole

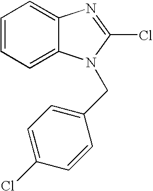

To a solution of 2-Chlorobenzimidazole (380 mg, 3 mmol) in dry DMF (10 ml) was added Sodium hydride (55%, 131 mg, 3 mmol). After stirring for 1 hour at RT, 4-Chlorobenzyl bromide (513 mg, 3 mmol) was added. The reaction mixture was stirred for 1 hour at 100° C. After cooling, the mixture was partitioned between EtOAc and water. The organic phase was dried over MgSO₄ and concentrated in vacuo. The oily residue was crystallized upon treatment with heptanes to give title compound Example 23 Step 1 (100 mg, 0.4 mmol, 15%)

### Step 2

Title compound Example 23 (80 mg, 0.15 mmol, 38%) was synthesized according to a procedure similar to the one described in Example 4 using N-{3-[1-(3-Amino-propyl)-piperidin-4-yl]-phenyl}-acetamide (Example 1 Step 2, 150 mg, 0.54 mmol) and 2-Chloro-1-(4-chloro-benzyl)-1H-benzoimidazole (Example 23 Step 1, 100 mg, 0.4 mmol).

¹H-NMR (300 MHz, CDCl₃); δ 1.38-1.49 (m, 2H), 1.76-2.01 (m, 5H), 2.21 (s, 3H), 2.41-2.54 (m, 4H), 3.02-3.05 (d, 2H), 3.67 (d, 2H), 5.11 (s, 2H), 6.58 (bs, 1H), 6.73-6.75 (d, 1H), 6.87-6.90 (d, 1H), 6.94-6.99 (t, 1H), 7.06-7.13 (m, 3H), 7.20-7.23 (m, 3H), 7.28-7.30 (1H), 7.43 (s, 1H), 7.48-7.51 (d, 1H), 7.90 (bs, 1H).

LCMS: Rt=3.83 min, 516, 518 m/z [M]⁺

### EXAMPLE 24

### N-[3-(1-{3-[1-(4-Nitro-benzyl)-1H-benzoimidazol-2-ylamino]-propyl}-piperidin-4-yl)-phenyl]-acetamide

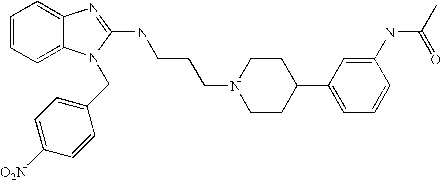

Title compound Example 24 (18.4 mg, 0.05 mmol, 14%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(4-nitro-benzyl)-1H-benzoimidazole (72 mg, 0.25 mmol, synthesized according to the procedure described in Example 23 Step 1).

LCMS: Rt=3.52 min, 527 m/z [M]⁺

### EXAMPLE 25

### N-[3-(1-{3-[1-(3-Trifluoromethyl-benzyl)-1H-benzoimidazol-2-ylamino]-propyl}-piperidin-4-yl)-phenyl]-acetamide

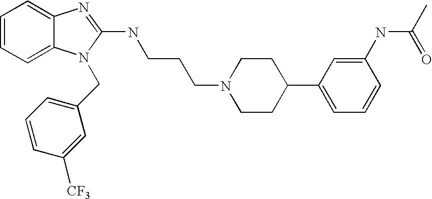

Title compound Example 25 (29.4 mg, 0.05 mmol, 21%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(3-trifluoromethyl-benzyl)-1H-benzoimidazole (77 mg, 0.25 mmol, synthesized according to the procedure described in Example 23 Step 1).

LCMS: Rt=4.04 min, 550 m/z [M]⁺

### EXAMPLE 26

### N-[3-(1-{3-[1-(4-tert-Butyl-benzyl)-1H-benzoimidazol-2-ylamino]-propyl}-piperidin-4-yl)-phenyl]-acetamide

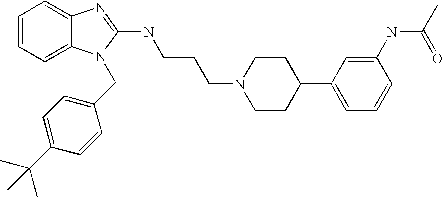

Title compound Example 26 (29.8 mg, 0.05 mmol, 22%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(4-tert-butyl-benzyl)-1H-benzoimidazole 82 mg, 0.25 mmol, synthesized according to the procedure described in Example 23 Step 1).

LCMS: Rt=4.37 min, 538 m/z [M]⁺

### EXAMPLE 27

### N-[3-(1-{3-[1-(4-Trifluoromethoxy-benzyl)-1H-benzoimidazol-2-ylamino]-propyl}-piperidin-4-yl)-phenyl]-acetamide

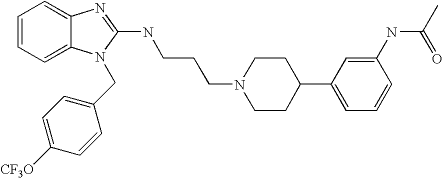

Title compound Example 27 (29.6 mg, 0.05 mmol, 20.9%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(4-trifluoromethoxy-benzyl)-1H-benzoimidazole (82 mg, 0.25 mmol, synthesized according to the procedure described in Example 23 Step 1).

LCMS: Rt=4.06 min, 566 m/z [M]⁺

### EXAMPLE 28

### N-[3-(1-{3-[1-(3-Trifluoromethoxy-benzyl)-1H-benzoimidazol-2-ylamino]-propyl}-piperidin-4-yl)-phenyl]-acetamide

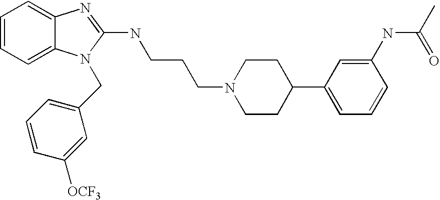

Title compound Example 28 (10.3 mg, 0.02 mmol, 7%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(3-trifluoromethoxy-benzyl)-1H-benzoimidazole (82 mg, 0.25 mmol, synthesized according to the procedure described in Example 23 Step 1).

LCMS: Rt=3.92 min, 566 m/z [M]⁺

### EXAMPLE 29

### N-[3-(1-{3-[1-(3-Methoxy-benzyl)-1H-benzoimidazol-2-ylamino]-propyl}-piperidin-4-yl)-phenyl]-acetamide

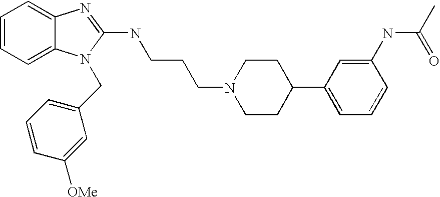

Title compound Example 29 (6.6 mg, 0.01 mmol, 5%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(3-methoxy-benzyl)-1H-benzoimidazole (70 mg, 0.25 mmol, synthesized according to the procedure described in Example 23 Step 1).

LCMS: Rt=3.46 min, 512 m/z [M]⁺

### EXAMPLE 30

### N-[3-(1-{3-[1-(3-Cyano-benzyl)-1H-benzoimidazol-2-ylamino]-propyl}-piperidin-4-yl)-phenyl]-acetamide

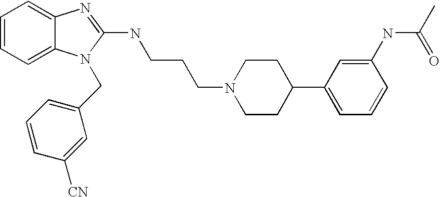

Title compound Example 30 (3.1 mg, 0.006 mmol, 2.3%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(3-cyano-benzyl)-1H-benzoimidazole (67 mg, 0.25 mmol, synthesized according to the procedure described in Example 23 Step 1).

LCMS: Rt=3.29 min, 507 m/z [M]⁺

### EXAMPLE 31

### N-[3-(1-{3-[1-(4-Cyano-benzyl)-1H-benzoimidazol-2-ylamino]-propyl}-piperidin-4-yl)-phenyl]-acetamide

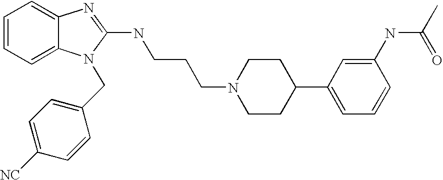

Title compound Example 31 (15.7 mg, 0.03 mmol, 12%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(4-cyano-benzyl)-1H-benzoimidazole (70 mg, 0.25 mmol, synthesized according to the procedure described in Example 23 Step 1).

LCMS: Rt=3.23 min, 507 m/z [M]⁺

### EXAMPLE 32

### N-[3-(1-{3-[1-(3-Chloro-benzyl)-1H-benzoimidazol-2-ylamino]-propyl}-piperidin-4-yl)-phenyl]-acetamide

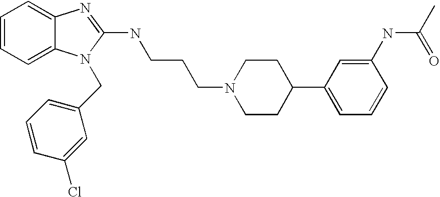

Title compound Example 32 (25.6 mg, 0.05 mmol, 10%) was synthesized according to a procedure similar to the one described in Example 4 using N-{3-[1-(3-Amino-propyl)-piperidin-4-yl]-phenyl}-acetamide (Example 1 Step 2, 100 mg, 0.36 mmol) and 2-Chloro-1-(3-chloro-benzyl)-1H-benzoimidazole (140 mg, 0.5 mmol, synthesized according to the procedure described in Example 23 Step 1).

LCMS: Rt=3.51 min, 516, 518 m/z [M]⁺

### EXAMPLE 33

### N-[3-(1-{3-[1-(3,4-Difluoro-benzyl)-1H-benzoimidazol-2-ylamino]-propyl}-piperidin-4-yl)-phenyl]-

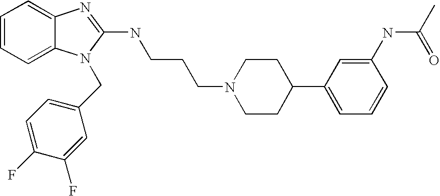

Title compound Example 33 (36 mg, 0.07 mmol, 13%) was synthesized according to a procedure similar to the one described in Example 4 using N-{3-[1-(3-Amino-propyl)-piperidin-4-yl]-phenyl}-acetamide (Example 1 Step 2, 200 mg, 0.7 mmol) and 2-Chloro-1-(3,4-difluoro-benzyl)-1H-benzoimidazole (140 mg, 0.5 mmol, synthesized according to the procedure described in Example 23 Step 1).

LCMS: Rt=3.58 min, 518 m/z [M]⁺

### Biological Results

### Materials and Methods

Transfections and Tissue Culture—The cDNA encoding the human MCH-1 receptor was cloned from a human brain cDNA library and cloned into the eukaryotic expression vector pcDNA3.1 (Invitrogen). Assays were performed on transiently transfected COS-7 cells or stably transfected CHO (Chinese Hamster Ovary) cells, expressing the human MCH-1 receptor in pcDNA3.1. Stable MCH-1 receptor transfectants of CHO cells were obtained using 5 μg, plasmid cDNA and a standard calcium phosphate transfection method (Johansen et al., 1990; Gether et al., 1992) with subsequent selection in 1 mg/ml G418 (Life Technology). Clones were screened by a MCH receptor radioligand binding assay (as described below). Stably transfected CHO cells were maintained in RPMI 1640 culture medium (Invitrogen), supplemented with 10% fetal calf serum (Invitrogen), 100 U/ml penicillin, 100 μg/ml streptomycin (Life Technology), and 500 μg/ml G418 (Life Technology). COS-7 cells were grown in Dulbecco's modified Eagle's medium (DMEM) 1885 (Invitrogen) supplemented with 10% fetal calf serum, 100 U/ml penicillin, 100 μg/ml streptomycin, and were transiently transfected by a standard calcium phosphate transfection method (Johansen et al., 1990; Gether et al., 1992) two days before assay.

Radioligand Binding Assay—Transiently transfected COS-7 cells or stably transfected CHO cells, expressing human MCH-1 receptor were seeded in multi-well culture plates one day before the assay. The number of cells per well was determined by the apparent expression efficiency of the cell line aiming at 5-10% binding of the added radioligand. Cells were assayed by competition binding for 3 hours at room temperature using 15 pM [¹²⁵I]-MCH (Amersham Pharmacia Biotech) plus variable amounts of unlabeled ligand in 0.5 ml of a 25 mM Hepes buffer, pH 7.4, supplemented with 10 mM MgCl₂, 5 mM MnCl₂, 10 mM NaCl, 0.1% (w/v) bovine serum albumin (BSA), 100 μg/ml bacitracin. The assay was performed in duplicate. Nonspecific-binding was determined as the binding in the presence of 1 μM MCH (Bachem). Binding data were analyzed and IC₅₀ values determined by non-linear regression using the Prism software (GraphPad software, San Diego). Values of the dissociation and inhibition constants (K_{d} and Kᵢ) were estimated from competition binding using the equations K_{d}=IC₅₀−L and Kᵢ=IC₅₀/(1+L/K_{d}), respectively, where L is the concentration of radioligand.

Scintillation Proximity Assay (SPA)—Measurement of [¹²⁵I]-MCH binding was performed in duplicates by incubating membranes and beads with tracer in the presence of various concentrations of test compounds (10⁻⁸ to 10⁻⁴ M) in DMSO (3 μl) at room temperature for two hours. Membranes and beads were pre-incubated for 20 min. The binding buffer contained 50 mM Tris (pH 7.4), 8 mM MgCl2, 12% glycerol, 0.1% (w/v) bovine serum albumin (BSA), and protease inhibitors (Complete protease inhibitor cocktail tablets, Roche). A final [¹²⁵I]-MCH (2000 Ci/mmol; Amersham Pharmacia Biotech) concentration of 75.000 cpm/well (33.8 nCi) was applied and PEI-treated WGA-coupled PVT SPA beads, type B from Amersham Pharmacia Biotech were used at a final concentration of 0.4 mg/well. Moreover, CHO-K1 membranes expressing the hMCH receptor were purchased from Euroscreen (ES-370-M) and a final concentration of 2 μg/well were used. Binding data were analyzed and IC₅₀ values determined by non-linear regression using the Prism software (GraphPad software, San Diego). Values of the inhibition constant (Kᵢ) were estimated from competition binding using the equation Kᵢ=IC₅₀/(1+L/K_{d}), where L and K_{d} are the concentration and affinity constant, respectively, of the radioligand.

### REFERENCES

Gether, U., Marray, T., Schwartz, T. W., and Johansen, T. E. (1992). Stable expression of high affinity NK₁ (substance P) and NK₂ (neurokinin A) receptors but low affinity NK₃ (neurokinin B) receptors in transfected CHO cells. FEBS Lett., 296, 241-244.
Johansen, T. E., Schøller, M. S., Tolstoy, S. and Schwartz, T. W. (1990). Biosynthesis of peptide precursors and protease inhibitors using new constitutive and inducible eukaryotic expressions vectors. FEBS Lett., 267, 289-294.

### Binding Affinity:

The compounds display binding affinity to the MCH1 receptor according to the following degrees:

IC₅₀<0.5 nM:

Examples: 1, 2, 3, 4, 5, 6, 7, 8, 9, 10, 11, 12 and 19

0.5 nM<IC₅₀<5 nM:

Examples: 13, 14, 15, 16, 17 and 18

5 nM<IC₅₀<10 nM:

Examples: 20, 21 and 22

1. A compound of formula (I), or a salt, hydrate or solvate thereof: 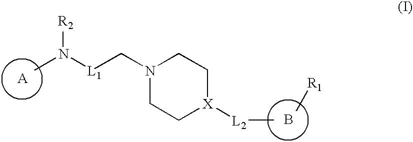
wherein
ring B is selected from 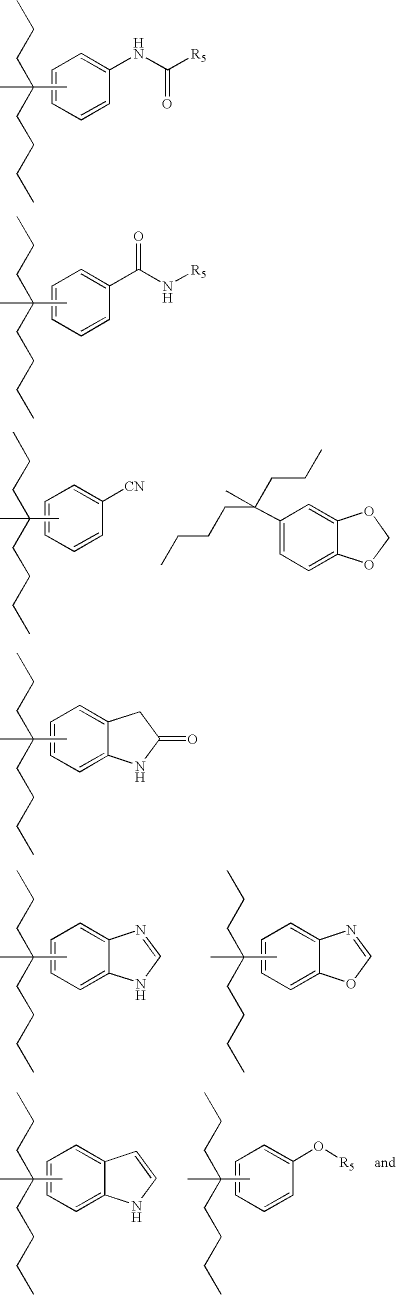 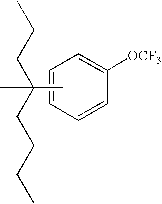
wherein R₅ is C₁-C₄ alkyl or cyclopropyl;
R₁ is attached to a ring carbon of ring B, and represents hydrogen, F, Cl, or —OCH₃;
X is ═CH— or ═N—;
L₁ is —CH₂— or —CH₂CH₂—;
L₂ is a bond, —CH₂— or —CO—;
R₂ is H or C₁-C₃ alkyl, or —N(R₂) L₁- is selected from 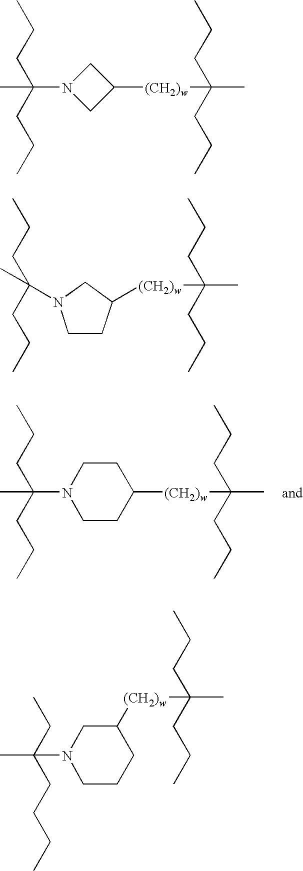
wherein w is 0 or 1;
ring A is selected from 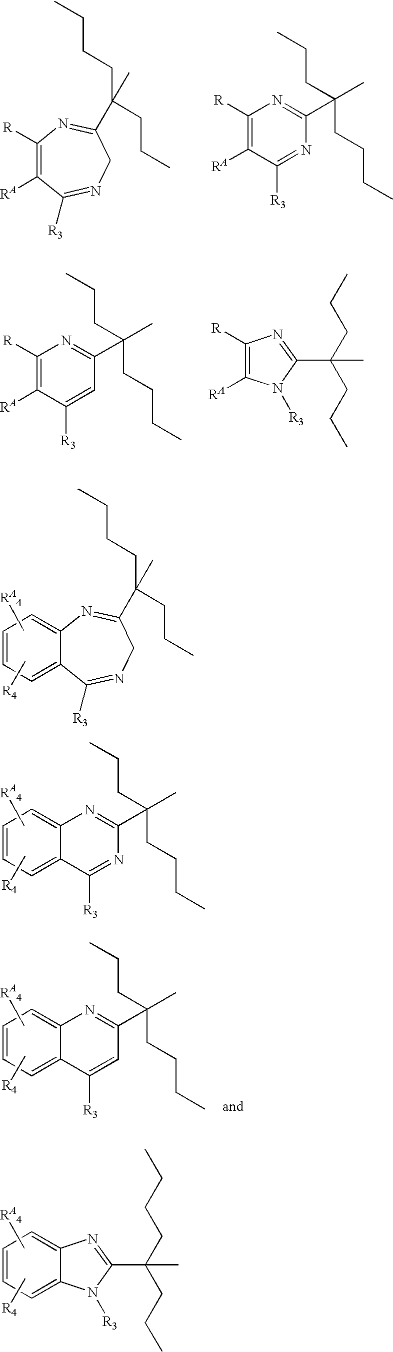
wherein R and R^{A} independently represent hydrogen, C₁-C₄ alkyl, C₁-C₄ alkoxy, halogen, —SCF₃, —OCF₃, or —CF₃;
R₄ and R^{A}₄ independently represent hydrogen, methyl, ethyl, methoxy, F, Cl, —CN, —OCF₃, —CF₃, —CONHCH₃, or —NHCOCH₃; or R₄ and R^{A}₄ together represent —O—CH₂—O— wherein the oxygens are attached to adjacent ring carbons; and
R₃ is a radical of formula -(Z)ₘ-(Alk¹)ₚ-Q wherein
Q is an optionally substituted monocyclic carbocyclic or heterocyclic ring of 5-, 6- or 7-ring atoms;
m and p are independently 0 or 1,
Alk¹ is optionally substituted straight or branched chain divalent C₁-C₃ alkylene radical which may contain a compatible —O—, —S— or —NR₇— link wherein R₇ is hydrogen, methyl, ethyl or n- or iso-propyl, and
Z is —O— or —NR₈—, wherein R₈ is hydrogen, methyl, ethyl or n- or iso-propyl.
PROVIDED THAT ring A is not 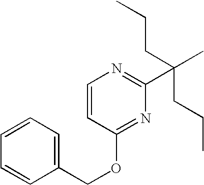
when R₂ is H, L₁ is —CH₂CH₂—, X is ═N—, L₂ is a bond, ring B is phenyl, and R₁ is o-methoxy.

2. A compound as claimed in claim 1 wherein the ring A is of formula (IA), (IB) or (IC):

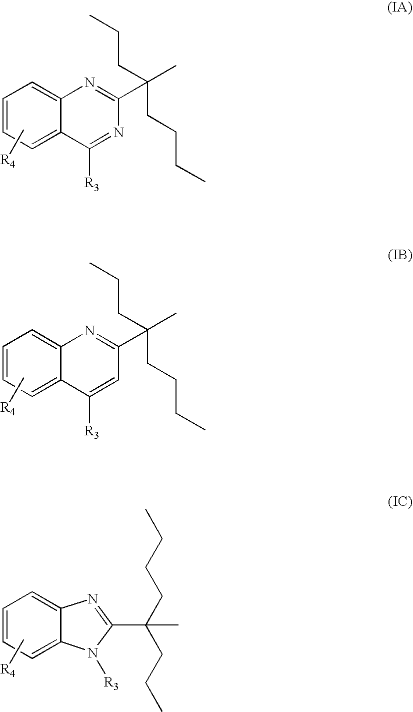

3. A compound as claimed in claim 1 wherein the ring A is of formula (IE) or (IF):

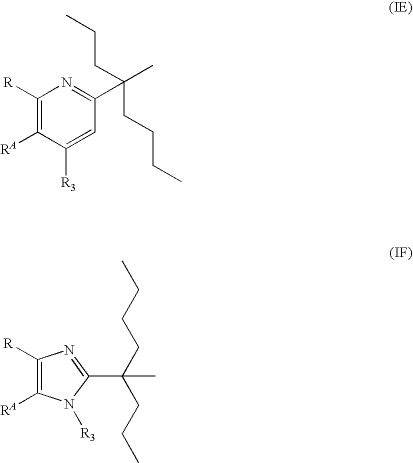

4. A compound as claimed in claim 1 wherein the ring A is of formula (ID) 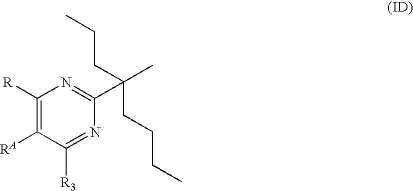
wherein, in the group R₃, -(Z)ₘ-(Alk¹)ₚ- is a bond.

5. A compound as claimed in claim 1 wherein, in ring A, neither of R^{A} and R is hydrogen.

6. A compound as claimed in claim 1 wherein, in ring A, R^{A}₄ is hydrogen.

7. A compound as claimed in claim 1 wherein ring B is of formula (IG), (IH) or (IK):

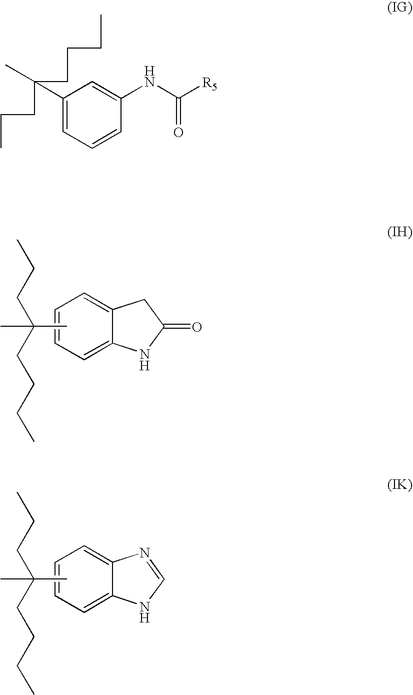

8. A compound as claimed in claim 1 wherein R₅ is methyl.

9. A compound as claimed in wherein L₂ is a bond.

10. A compound as claimed in claim 1 wherein, in the group R₃, m and p are both 0; or m is 1 and p is 0; or m is 0 and p is 1 and -Alk¹- is —CH₂—.

11. A compound as claimed in claim 1 wherein, in the group R₃, m is 0 and p is 1 and Alk¹ is —C(═O)—.

12. A compound as claimed in claim 10 wherein m is 1 and p is 0 and ring A is selected from

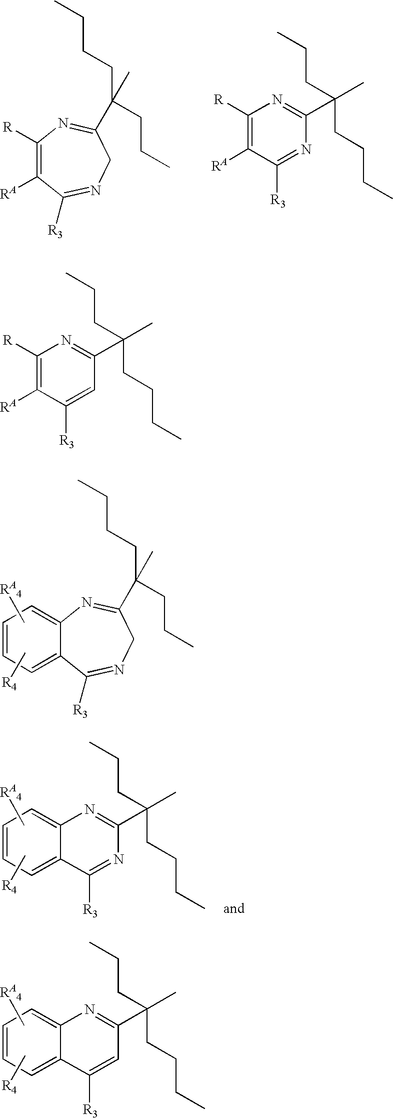

13. A compound as claimed in claim 1 wherein, in the group R₃, Q is an aryl or heteroaryl ring.

14. A compound as claimed in claim 1 wherein, in the group R₃, Q is optionally substituted phenyl, pyridyl, or thienyl.

15. A compound as claimed in claim 14 wherein Q is phenyl, optionally substituted by fluoro, chloro, methyl, —CN, —OCF₃, —CF₃, —SCH₃, —SO₂CH₃, —SO₂NH₂, —SO₂NHCH₃, —CONHCH₃ or methoxy.

16. A compound as claimed in claim 14 wherein R₃ is phenyl, mono-substituted in the 4-position by fluoro, chloro, methyl, —CN, —OCF₃, —CF₃, —SCH₃, —SO₂CH₃, —SO₂NH₂, —SO₂NHCH₃, —CONHCH₃ or methoxy.

17. A compound as claimed in claim 15 wherein, in the group R₃, m and p are both 0, or m is 0 and p is 1 and Alk¹ is —CH₂—.

18. A compound as claimed in claim 1 having the formula: 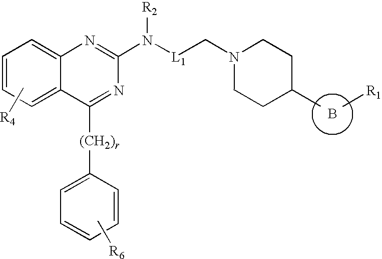
wherein r is 0 or 1, and R₆ is fluoro, chloro, methyl, —CN, —OCF₃, —CF₃, —SCH₃, —SO₂CH₃, —SO₂NH₂, —SO₂NHCH₃, —CONHCH₃ or methoxy.

19. A compound as claimed in claim 1 having the formula: 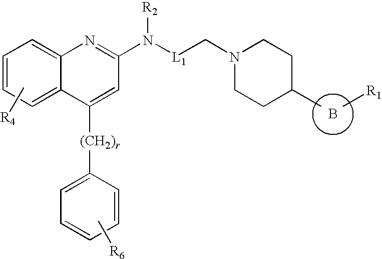
wherein r is 0 or 1, and R₆ is fluoro, chloro, methyl, —CN, —OCF₃, —CF₃, —SCH₃, —SO₂CH₃, —SO₂NH₂, —SO₂NHCH₃, —CONHCH₃ or methoxy.

20. A compound as claimed in claim 1 having the formula: 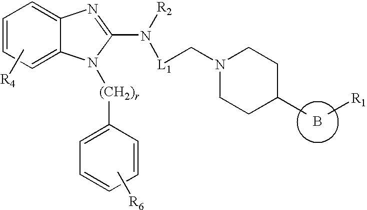
wherein r is 0 or 1, and R₆ is fluoro, chloro, methyl, —CN, —OCF₃, —CF₃, —SCH₃, —SO₂CH₃, —SO₂NH₂, —SO₂NHCH₃, —CONHCH₃ or methoxy.

21. A compound as claimed in claim 1 wherein R₂ is hydrogen.

22. A compound as claimed in claim 1 wherein R₆ is in the 4-position of the phenyl ring.

23. A compound as claimed in claim 1 wherein R₁ is hydrogen.

24. A compound as claimed in claim 1 having one of the following structural formulae:

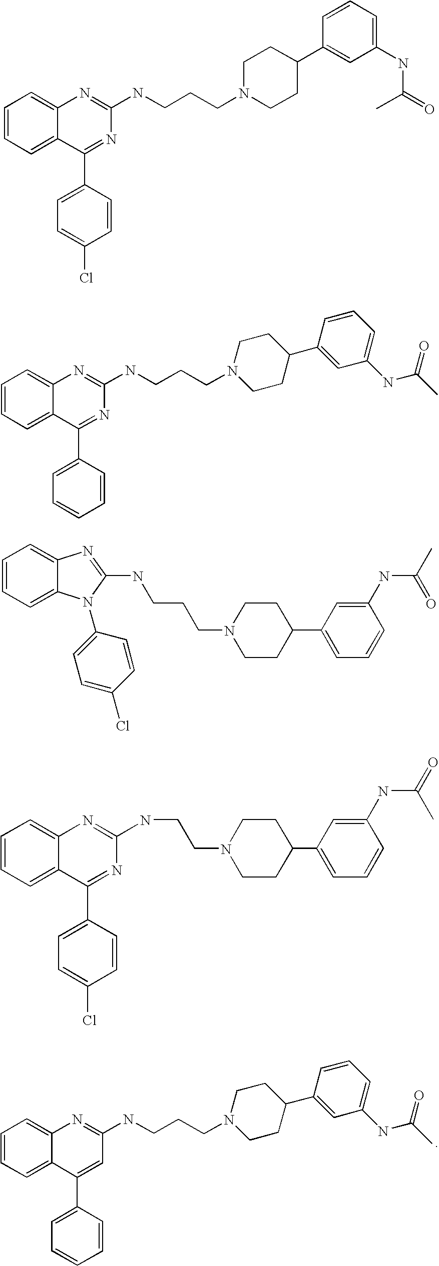

25. A compound as claimed in claim 1 having one of the following structural formulae:

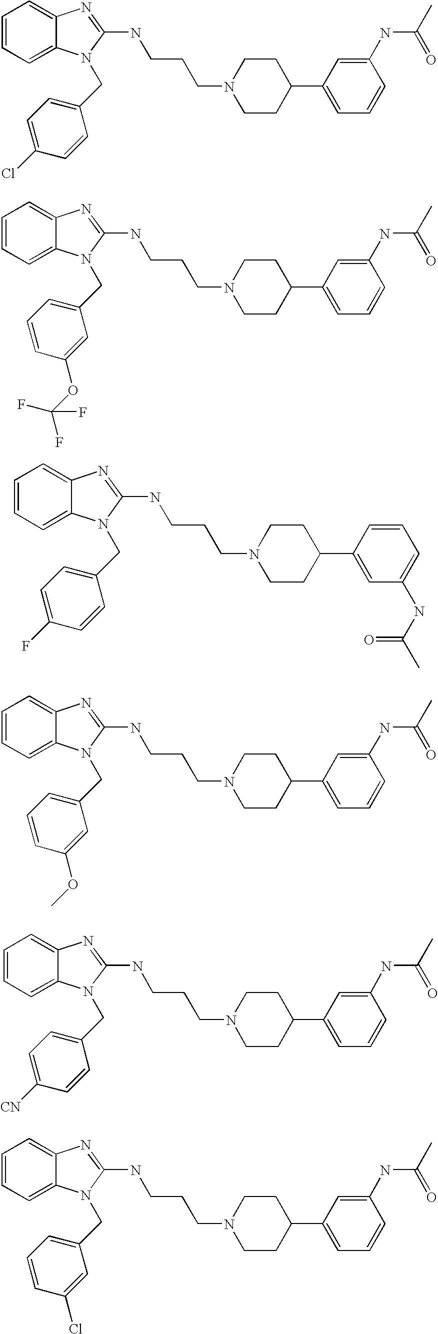

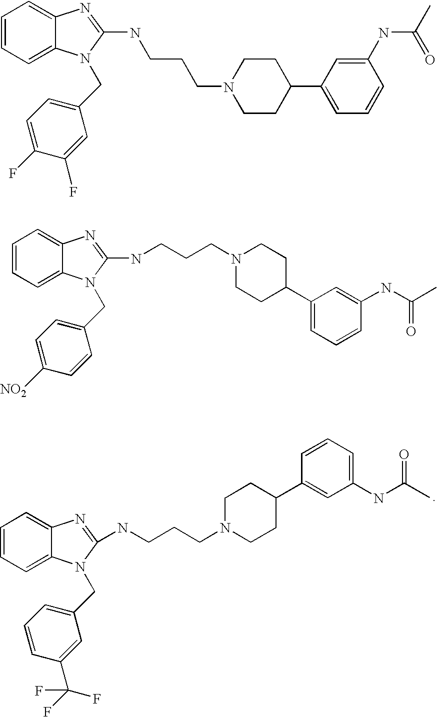

26. A pharmaceutical composition comprising a compound as claimed claim 1, and a pharmaceutically acceptable carrier.

27. A pharmaceutical composition comprising a compound as claimed in claim 1 in an effective amount for the treatment of a disorder responsive to modulation of MCH activity.

28. A method of treatment of a mammalian subject suffering from a disorder responsive to modulation of MCH activity, comprising administering to the subject an effective amount of a compound as claimed in claim 1.

29. The method as claimed in claim 28, wherein the disorder is obesity, metabolic syndrome, Type II diabetes, bulimia, depression, anxiety, psychosis, dementia, a mood disorder, a cognitive disorder, stress, memory impairment, an abuse disorder, or a mentally-based sexual function disorder.

30. A pharmaceutical composition comprising a compound as claimed in claim 1 in an effective amount for modifying the feeding behaviour of a mammal.

31. A method of treatment of a mammalian subject to modify the feeding behaviour of the subject, comprising administering to the subject an effective amount of a compound as claimed in claim 1.

32. A pharmaceutical composition comprising a compound as claimed in claim 1 in an effective amount for modifying reducing the body mass of a mammal.

33. A method of treatment of a mammalian subject to reduce the body mass of the subject, comprising administering to the subject an effective amount of a compound as claimed in claim 1.
